SEQUENCE LISTING (37 sequences)

SEQ ID NO: 1 - DNA, 35 bp, synthetic construct
  source 1..35: mol_type = other DNA
gactcgagtcgacatcgattttttttttttttttt

SEQ ID NO: 2 - DNA, 21 bp, synthetic construct
  source 1..21: mol_type = other DNA
catcaggatcatcagcgattc

SEQ ID NO: 3 - DNA, 20 bp, synthetic construct
  source 1..20: mol_type = other DNA
accagacaaagatgtctgtg

SEQ ID NO: 4 - DNA, 24 bp, synthetic construct
  source 1..24: mol_type = other DNA
cctatcagtgatgttgtccttagc

SEQ ID NO: 5 - DNA, 23 bp, synthetic construct
  source 1..23: mol_type = other DNA
caattctatatgagtcacctgag

SEQ ID NO: 6 - DNA, 22 bp, synthetic construct
  source 1..22: mol_type = other DNA
agagtagtccttccgcttatag

SEQ ID NO: 7 - DNA, 22 bp, synthetic construct
  source 1..22: mol_type = other DNA
tgtgacctattctaacgacaag

SEQ ID NO: 8 - DNA, 21 bp, synthetic construct
  source 1..21: mol_type = other DNA
gaaatgttgttgggtttgctc

SEQ ID NO: 9 - DNA, 19 bp, synthetic construct
  source 1..19: mol_type = other DNA
cgtcaggttcaggtggtac

SEQ ID NO: 10 - DNA, 21 bp, synthetic construct
  source 1..21: mol_type = other DNA
ggtttagtgcagggtcttatc

SEQ ID NO: 11 - DNA, 24 bp, synthetic construct
  source 1..24: mol_type = other DNA
agggtcttatcctctgctatggag

SEQ ID NO: 12 - DNA, 24 bp, synthetic construct
  source 1..24: mol_type = other DNA
cttgctaaaataccggtttgaatc

SEQ ID NO: 13 - DNA, 21 bp, synthetic construct
  source 1..21: mol_type = other DNA
tccaattctgctatggaagag

SEQ ID NO: 14 - DNA, 23 bp, synthetic construct
  source 1..23: mol_type = other DNA
gagggaggtatgtagaggggttc

SEQ ID NO: 15 - DNA, 21 bp, synthetic construct
  source 1..21: mol_type = other DNA
tgccagggaaatttacgaatc

SEQ ID NO: 16 - DNA, 22 bp, synthetic construct
  source 1..22: mol_type = other DNA
acgtgaatttaaagaatccaac

SEQ ID NO: 17 - DNA, 22 bp, synthetic construct
  source 1..22: mol_type = other DNA
aggtattgtcactccagcttac

SEQ ID NO: 18 - DNA, 24 bp, synthetic construct
  source 1..24: mol_type = other DNA
ctacctgtgcaaatgtaatatctg

SEQ ID NO: 19 - DNA, 20 bp, synthetic construct
  source 1..20: mol_type = other DNA
aggacctagaaaattaccag

SEQ ID NO: 20 - DNA, 22 bp, synthetic construct
  source 1..22: mol_type = other DNA
gcaatactatcctatacacatg

SEQ ID NO: 21 - DNA, 22 bp, synthetic construct
  source 1..22: mol_type = other DNA
atcatgcatccgctgtaattag

SEQ ID NO: 22 - DNA, 20 bp, synthetic construct
  source 1..20: mol_type = other DNA
aaaacttaggaatccatgtt

SEQ ID NO: 23 - DNA, 20 bp, synthetic construct
  source 1..20: mol_type = other DNA
ccacaaaatgacctccagta

SEQ ID NO: 24 - DNA, 21 bp, synthetic construct
  source 1..21: mol_type = other DNA
caccattgcctttcagagttg

SEQ ID NO: 25 - DNA, 29 bp, synthetic construct
  source 1..29: mol_type = other DNA
gccatattttgtggaataathathaaygg

SEQ ID NO: 26 - protein, 26 aa, synthetic construct
CTCATTTTGTAIGTCATYTTNGCRAA

SEQ ID NO: 27 - DNA, 21 bp, synthetic construct
  source 1..21: mol_type = other DNA
gctctcgaagattagtcaatg

SEQ ID NO: 28 - DNA, 24 bp, synthetic construct
  source 1..24: mol_type = other DNA
caagattccatgttagacctaatg

SEQ ID NO: 29 - DNA, 23 bp, synthetic construct
  source 1..23: mol_type = other DNA
agactatatgagagattgaactc

SEQ ID NO: 30 - DNA, 20 bp, synthetic construct
  source 1..20: mol_type = other DNA
ggatgcttatttatctgatc

SEQ ID NO: 31 - DNA, 21 bp, synthetic construct
  source 1..21: mol_type = other DNA
ccagacaaagaaagctatagg

SEQ ID NO: 32 - DNA, 22 bp, synthetic construct
  source 1..22: mol_type = other DNA
gtgtattgagttggtaatattc

SEQ ID NO: 33 - DNA, 21 bp, synthetic construct
  source 1..21: mol_type = other DNA
gatagcgtaatataacaggtg

SEQ ID NO: 34 - protein, 24 aa, synthetic construct
MRTGSERFIGAVIAGVALGVATAA

SEQ ID NO: 35 - protein, 28 aa, synthetic construct
QSVASSRRHKRFAGVVLAGAALGVATAA

SEQ ID NO: 36 - protein, 28 aa, synthetic construct
QSVGSGRRQRRFAGVVLAGAALGVATAA

SEQ ID NO: 37 - protein, 28 aa, synthetic construct
MRTGSGRRQRRFIGAVIAGVALGVATAA

(19) United States
(12) Patent Application Publication
(10) Pub. No.: US 2025/0304921 A1
(43) Pub. Date: Oct. 2, 2025

(54) REVERSE GENETICS SYSTEM FOR FELINE MORBILLIVIRUS

(71) Applicant: University of Pittsburgh-Of The Commonwealth System of Higher Education, Pittsburgh, PA (US)

(72) Inventors: William Paul Duprex, Pittsburgh, PA (US); Shamkumar Nambulli, Pittsburgh, PA (US); Linda J. Murphy, Pittsburgh, PA (US)

(73) Assignee: University of Pittsburgh-Of The Commonwealth System of Higher Education, Pittsburgh, PA (US)

(21) Appl. No.: 18/869,678

(22) PCT Filed: May 26, 2023
(86) PCT No.: PCT/US2023/067540

§ 371 Date: Nov. 26, 2024

Related U.S. Application Data
(60) Provisional application No. 63/346,672, filed on May 27, 2022.
(60) Provisional application No. 63/504,364, filed on May 25, 2023.

(51) Int. Cl. C12N 7/00; C12N 15/10; C12N 15/85; C12Q 1/6809; C12Q 1/686; C12Q 1/6869; C12Q 1/70

(52) U.S. Cl. CPC C12N 7/00; C12N 15/1096; C12N 15/85; C12Q 1/6809; C12Q 1/686; C12Q 1/6869; C12Q 1/701; C12N 2710/24144; C12N 2760/18452; C12N 2800/107

(57) ABSTRACT

Disclosed are methods of producing recombinant feline morbillivirus (FeMV) using reverse genetics. In some aspects, the methods comprise: (a) extracting FeMV RNA from an isolated FeMV positive sample, (b) generating cDNAs from the FeMV RNA using primers that specifically hybridize to the FeMV RNA, (c) generating cDNA PCR amplicons from the cDNAs using primers that specifically hybridize to the cDNAs to produce cDNA PCR amplicons, (d) amplifying genomic and antigenomic termini of the FeMV RNA by rapid amplification of cDNA ends (RACE) using one or more RACE primers to produce RACE PCR amplicons, (e) purifying the cDNA PCR amplicons of step (c) and the RACE PCR amplicons of step (d) to produce purified DNA, (f) sequencing the purified DNA to produce consensus sequences, (g) assembling the consensus sequences to produce a full-length FeMV genome, and (h) assembling the full-length FeMV genome in a plasmid.

### CROSS REFERENCE TO RELATED APPLICATIONS

This patent application claims the benefit of U.S. Provisional Patent Application No. 63/346,672 filed May 27, 2022, and U.S. Provisional Patent Application No. 63/504,364 filed May 25, 2023, the disclosures of which are incorporated by reference in their entireties herein.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 33,665 Byte XML file named “767393.XML,” created May 26, 2023.

### BACKGROUND OF THE INVENTION

Feline morbillivirus (FeMV) is a negative sense, single stranded, non-segmented RNA virus, first discovered in domestic cats in Hong Kong and China in 2012. FeMV has been postulated to be a causative agent in feline chronic kidney disease (CKD), the leading cause of morbidity and mortality in older cats. There is a lack of effective treatments available for FeMV and CKD. Producing recombinant FeMV is an important step towards understanding and treating FeMV infections and CKD. Accordingly, there is a need for methods of producing recombinant FeMV.

### BRIEF SUMMARY OF THE INVENTION

An aspect of the invention provides methods of producing recombinant FeMV, the methods comprising using reverse genetics.

An aspect of the invention further provides methods of producing recombinant FeMV, the methods comprising: (a) extracting FeMV RNA from an isolated FeMV positive sample, (b) generating cDNAs from the FeMV RNA using primers that specifically hybridize to the FeMV RNA, (c) generating cDNA PCR amplicons from the cDNAs using primers that specifically hybridize to the cDNAs to produce cDNA PCR amplicons, (d) amplifying genomic and antigenomic termini of the FeMV RNA by rapid amplification of cDNA ends (RACE) using one or more RACE primers to produce RACE PCR amplicons, (e) purifying the cDNA PCR amplicons of step (c) and the RACE PCR amplicons of step (d) to produce purified DNA, (f) sequencing the purified DNA to produce consensus sequences, (g) assembling the consensus sequences to produce a full-length FeMV genome, and (h) assembling the full-length FeMV genome in a plasmid.

### BRIEF DESCRIPTION OF THE SEVERAL VIEWS OF THE DRAWING(S)

FIG. 1A is a drawing showing a summary of a dual bimolecular complementation assay. CRFK-feCD150 (or CFRK-hCD150 or CRFK for controls) and CRFK cell populations are transfected with pCG-rlucEGFPC and pCG-NrlucEGFP respectively. CRFK cells are simultaneously transfected with FeMV, MV, or CDV F and H glycoproteins (or F/G for Nipah virus). After 24 hours the cell populations are trypsinised and mixed. Glycoprotein induced fusion of the cell populations results in rLuc-EGFP complementation leading to EGFP fluorescence and renilla luciferase activity.

FIG. 1B is a bar graph showing relative luminescence detected when CRFK cells expressing various viral F and H/G glycoprotein pairs and NrlucEGFP are mixed with CRFK cells, CRFK-feCD150 or CRFK-hCD150 cells expressing rlucEGFPC and triplicate cell monolayers are lysed and assayed for luciferase activity. Luminescence produced when CRFK cells expressing the glycoprotein pairs are mixed with CRFK-feCD150 cells are set to 100%.

FIG. 2A shows alignment of the predicted cleavage site and surrounding sequences of the FeMV^{US5} F protein (SEQ ID NO: 34) with the equivalent regions of MV^{KS} (SEQ ID NO: 35) and CDV^{RI} F proteins (SEQ ID NO: 36), and a modified FeMV^{US5} F protein engineered to contain a polybasic cleavage signal (FeMV^{US5}F_{PB}; SEQ ID NO: 37). The polybasic cleavage signal is boxed and an arrow marks the predicted cleavage site in F₀ which gives rise to F₁ and F₂ subunits.

FIG. 2B is a gel showing a Western blot analysis of transfected CRFK-feCD150 cell lysates. Cells were transfected with pCG-FeMV^{US5}F (lane 1) as a background blotting control, or pCG-FeMV^{US5}F_{AU1} (lanes 2-3) or pCG-FeMV^{US5}F_{PB-AU1} (lanes 4-5). Transfected cells were treated with DMSO (lanes 1, 2 and 4) or E64d cysteine protease inhibitor (lanes 3 and 5). F proteins were detected with anti-AU1 antibody (red) and an anti-β-actin antibody was used as a loading control (β; 42 kDa; green). The predicted F₀ (61 kDa) and F₁ (49 kDa) subunits are indicated based on the marker sizes (lane M).

FIG. 2C is a bar graph showing relative luminescence detected when CRFK cells expressing various viral F and H (or F/G for Nipah virus) glycoprotein pairs and NrlucEGFP are mixed with CRFK-feCD150 cells expressing rlucEGFPC in the presence of DMSO, furin inhibitor I, or E64d cysteine protease inhibitor and triplicate cell monolayers are lysed and assayed for luciferase activity. Luminescence produced when fusion assays were carried out in the presence of DMSO are set to 100%. FIGS. 2A-2C show FeMV glycoprotein-induced cell-to-cell fusion is inhibited by a cysteine protease inhibitor.

FIG. 3A is a schematic showing a representation of plasmids generated to allow recovery of rFeMV^{US5} expressing Venus from an additional transcription unit between the H and L genes (pFeMV^{US5}Venus (6); top) or P and M genes (pFeMV^{US5}Venus (3); bottom). Coding sequences (N, P/V/C, M, F, H, and L, dark gray), non-coding sequences (white), and intergenic trinucleotide (vertical black lines in non-coding sequences) are indicated. Genomes are surrounded by a T7 RNA polymerase promoter (T7, light gray), a hammerhead ribozyme (box to the immediate right of T7) a hepatitis delta ribozyme (δ, light gray) and T7 RNA polymerase terminator (φ, light gray) sequences to allow recovery of RNA corresponding to the viral genomes from the plasmids.

FIG. 3B is a graph showing the results from a multistep growth analysis of rFeMV^{US5}Venus (6) (pos 6) and rFeMV^{US5}Venus (3) (pos 3) in CRFK-feCD150 cells over 4 days. Error bars represent one standard error of the mean for all curves (n=3 for each virus).

FIG. 3C is a graph showing gaussia luciferase activity in HEp-2 cells at 2 days post-transfection with p(−)FeMV^{US5}DIGluc (DIGluc; rule-of-six compliant) or p(−)FeMV^{US5}DIGluc+3 (DIGluc+3; non rule-of-six compliant), and pCG-FeMV^{US5}N and pCG-FeMV^{US5}P either with (+L) or without (−L) pCG-FeMV^{US5}L.

FIG. 3D is a representative photomicrographs depicting Venus fluorescence observed in CRFK-feCD150 cells 5 days after infection with rFeMV^{US5}Venus (6) in the presence of DMSO.

FIG. 3E is a representative photomicrographs depicting Venus fluorescence observed in CRFK-feCD150 cells 5 days after infection with rFeMV^{US5}Venus (6) in the presence of furin inhibitor I.

FIG. 3F is a representative photomicrographs depicting Venus fluorescence observed in CRFK-feCD150 cells 5 days after infection with rFeMV^{US5}Venus (6) in the presence of inhibitor E64d.

FIG. 3G is a representative photomicrographs depicting Venus fluorescence observed in CRFK-feCD150 cells 5 days after infection with rFeMV^{US5}Venus (6) in the presence of inhibitor CA-074Me.

FIG. 3H is a bar graph showing luminescence (y-axis, relative light units; R.L.U.) detected 2 days after CRFK-feCD150 cells transfected with pCG-NrlucEGFP and infected with rFeMV^{US5}Venus (6) in the presence of inhibitors were overlaid (in the presence of inhibitors) with a population of CRFK-feCD150 cells that had previously been transfected with pCG-rlucEGFPC.

FIG. 3I is a bar graph showing quantification of virus recovered 4 days after infection of triplicate CRFK-feCD150 monolayers with rFeMV^{US5}Venus (6) or rFeMV^{US5}Venus (3) in the presence of DMSO, E64d cysteine protease inhibitor or CA-074Me cathepsin inhibitor. Titers determined in the presence of DMSO are set to 100%. FIGS. 3A-3J show rFeMV^{US5} induced cell-to-cell fusion is inhibited by a cathepsin protease inhibitor.

FIG. 4A is a set of graphs showing the body temperature of cats following infection with rFeMV^{US5}Venus (6) and rFeMV^{US5}Venus (3). The temperature of the cats was measured every 10 minutes pre-infection (grey) and post-infection (black) using an intraperitoneal data logger. Temperature data for cats euthanized at 7, 14, and 28 days post-infection (d.p.i.) are depicted left to right. * denotes procedure related temperature spikes.

FIG. 4B is a graph showing lymphocyte numbers measured in EDTA blood samples.

FIG. 4C is a graph showing the number of Venus positive cells as quantified by flow cytometry in purified white blood cell samples.

FIG. 4D is a graph showing the number of Venus positive cells in cell samples dissociated from mandibular (man.), retro-pharyngeal (RP) lymph nodes, and cervical (cerv.) lymph nodes (LN) or prepared from bronchoalveolar lavage (BAL) samples at 7 (D7) or 14 (D14) d.p.i.

FIG. 4E is an image of stained cat lung tissue showing virus distribution in cat lung tissue at 7 d.p.i. Visualized by immunodetection (IHC) of Venus protein in formalin fixed interstitial tissue sections. Scale bar (lower right) represents 200 μm.

FIG. 4F is an image of stained cat lung tissue showing virus distribution in cat lung tissue at 7 d.p.i. Visualized by immunodetection (IHC) of Venus protein in formalin fixed peri-bronchial tissue sections. Scale bar (lower right) represents 200 μm.

FIG. 4G is an image of stained cat lung tissue showing virus distribution in cat lung tissue at 7 d.p.i. Visualized by immunodetection (IHC) of Venus protein in formalin fixed bronchus associated lymphoid tissue sections. Scale bar (lower right) represents 200 μm.

FIG. 4H is an image of stained cat lung tissue showing virus distribution in cat lung tissue at 7 d.p.i. Visualized by detection of virus genome (RNAscope) by in situ hybridization in formalin fixed interstitial tissue sections. Scale bar (lower right) represents 200 μm.

FIG. 4I is an image of stained cat lung tissue showing virus distribution in cat lung tissue at 7 d.p.i. Visualized by detection of virus genome (RNAscope) by in situ hybridization in formalin fixed peri-bronchial tissue sections. Scale bar (lower right) represents 200 μm.

FIG. 4J is an image of stained cat lung tissue showing virus distribution in cat lung tissue at 7 d.p.i. Visualized by detection of virus genome (RNAscope) by in situ hybridization in formalin fixed bronchus associated lymphoid tissue sections. Scale bar (lower right) represents 200 μm.

FIG. 4K is an image showing detection of infected cells in renal medullary tubules at 28 d.p.i. Visualized by immunodetection (IHC) of Venus protein in serial sections. Scale bar (lower right corner) represents 100 μm.

FIG. 4L is an image detection of infected cells in renal medullary tubules at 28 d.p.i. Visualized by detection of virus genome (RNAscope) by in situ hybridization in serial sections. Scale bar (lower right corner) represents 100 μm.

FIG. 5A is a graph showing the body temperature of cats as measured every 5 minutes using a subcutaneous data logger. Temperatures for each d.p.i. were averaged and are displayed as the change in temperature relative to the average temperature for the seven days pre-infection (plotted as 0 d.p.i.).

FIG. 5B is a graph showing the percentage of Venus positive cells that were quantified by flow cytometry in purified white blood cell samples.

FIG. 5C is an image of a macroscopic detection of Venus fluorescence in thymus (gray arrow) at 7 d.p.i. FIG. 5C depicts an animal before removal of the thymus and upper respiratory tract.

FIG. 5D is an image of a macroscopic detection of Venus fluorescence in thymus at 7 d.p.i.

FIG. 5E is an image of a macroscopic detection of Venus fluorescence in tonsils (white arrows; tongue and tonsils shown at lower magnification in the inset for orientation) at 7 d.p.i.

FIG. 5F is an image of a macroscopic detection of Venus fluorescence in lymph nodes (representative lymph nodes marked with white arrows) at 7 d.p.i. FIG. 5F depicts the same animal as in FIG. 5C following removal of the thymus and upper respiratory tract.

FIG. 5G is an image of an infected cell (Venus positive) phenotyping using antibodies against CD20 (B cells), and myeloid/histiocyte antigen (monocytes/macrophages) in lung. The areas marked by white boxes in the main triple overlay image are shown at higher magnification as monocytes/macrophages/B cells and Venus positive/B cells double overlay images in the insets. Nuclei are counterstained with DAPI. Scale bar (lower right corner) represents 200 μm.

FIG. 5H is an image of an infected cell (Venus positive) phenotyping using antibodies against CD20 (B cells), and myeloid/histiocyte antigen (monocytes/macrophages) in tonsil. The areas marked by white boxes in the main triple overlay image are shown at higher magnification as monocytes/macrophages/B cells and Venus positive/B cells double overlay images in the insets. Nuclei are counterstained with DAPI. Scale bar (lower right corner) represents 200 μm.

FIG. 5I is an image of an infected cell (Venus positive) phenotyping using antibodies against CD20 (B cells), and myeloid/histiocyte antigen (monocytes/macrophages) in mesenteric lymph node. The areas marked by white boxes in the main triple overlay image are shown at higher magnification as monocytes/macrophages/B cells and Venus positive/B cells double overlay images in the insets. Nuclei are counterstained with DAPI. Scale bar (lower right corner) represents 200 μm.

FIG. 5J is an image of an infected cell (Venus positive) phenotyping using antibodies against CD20 (B cells), and myeloid/histiocyte antigen (monocytes/macrophages) in inguinal lymph node. The areas marked by white boxes in the main triple overlay image are shown at higher magnification as monocytes/macrophages/B cells and Venus positive/B cells double overlay images in the insets. Nuclei are counterstained with DAPI. Scale bar (lower right corner) represents 200 μm.

FIG. 5K is an image of an infected cell (Venus positive) phenotyping using antibodies against CD20 (B cells), and myeloid/histiocyte antigen (monocytes/macrophages) in axillary lymph node. The areas marked by white boxes in the main triple overlay image are shown at higher magnification as monocytes/macrophages/B cells and Venus positive/B cells double overlay images in the insets. Nuclei are counterstained with DAPI. Scale bar (lower right corner) represents 200 μm.

FIG. 6 is a set of images showing FeMV glycoproteins can use feCD150. Representative photomicrographs depicting EGFP fluorescence observed when CRFK cells expressing various viral F and H (or F/G for Nipah virus) glycoprotein pairs and NrlucEGFP are mixed with CRFK cells (top panels), CRFK-feCD150 (middle panels) or CRFK-hCD150 cells (bottom panels) expressing rlucEGFPC. Scale bars represent 200 μm.

FIG. 7 is a set of images showing FeMV glycoprotein-induced cell-to-cell fusion is inhibited by a cysteine protease inhibitor. Representative photomicrographs depicting EGFP fluorescence observed when CRFK cells expressing various viral F and H (or F/G for Nipah virus) glycoprotein pairs and NrlucEGFP are mixed with CRFK-feCD150 cells expressing rlucEGFPC in the presence of DMSO (top panels), furin inhibitor I (middle panels) or E64d cysteine protease inhibitor (bottom panels). Scale bars (lower right corner) represent 200 μm.

FIG. 8A is an image of a representative photomicrographs depicting Venus fluorescence observed in primary syncytia after recovery of rFeMV^{US5}Venus (6) from cloned cDNA in CRFK-feCD150 cells. Scale bar (lower right corner) represents 200 μm.

FIG. 8B is an image of a representative photomicrographs depicting Venus fluorescence observed in primary syncytia after recovery of rFeMV^{US5}Venus (3) from cloned cDNA in CRFK-feCD150 cells. Scale bar (lower right corner) represents 200 μm.

FIG. 9A is a representative set of higher magnification images of virus distribution in cat lung tissue at 7 d.p.i. Visualized by immunodetection of Venus protein in formalin fixed interstitial tissue sections. Rare infected pneumocytes (arrows point to the same cell in the main image and the higher magnification inset image) were observed. Scale bar (lower right corner) represents 100 μm.

FIG. 9B is a representative higher magnification image of virus distribution in cat lung tissue at 7 d.p.i. Visualized by immunodetection of Venus protein in formalin fixed submucosal bronchiole tissue sections. Scale bar (lower right corner) represents 100 μm.

FIG. 9C is a representative higher magnification image of virus distribution in cat lung tissue at 7 d.p.i. Visualized by immunodetection of Venus protein in formalin fixed bronchus associated lymphoid tissue sections. Scale bar (lower right corner) represents 100 μm.

FIG. 10A is a set of images showing rFeMV^{US5} targets monocytes/macrophages in secondary lymphoid tissues. Infected cell (Venus positive) phenotyping using antibodies against CD20 (B cells), and myeloid/histiocyte antigen (monocytes/macrophages) in axillary lymph node. The three individual (Venus, B-cell and monocyte/macrophage) channels are shown at low magnification along with the resultant merged image. The areas marked by white dashed boxes in the merge triple overlay image are shown at higher magnification in the image below. Nuclei were counterstained with DAPI. Scale bars represent 200 μm.

FIG. 10B is a set of images showing rFeMV^{US5} targets monocytes/macrophages in lung. Infected cell (Venus positive) phenotyping using antibodies against CD20 (B cells), and myeloid/histiocyte antigen (monocytes/macrophages) in lung. The three individual (Venus, B-cell and monocyte/macrophage) channels are shown at low magnification along with the resultant merged image. The areas marked by white dashed boxes in the merge triple overlay image are shown at higher magnification in the image below. Nuclei are counterstained with DAPI (grey). Scale bars represent 200 μm except for bottom panel which is 50 μm.

FIG. 11 is a set of images of photomicrographs showing rFeMV^{US5} infection of different cell types. Vero, Vero-feCD150, CRFK and CRFK-feCD150 cells were infected with rFeMV^{US5}Venus (6) at a multiplicity of infection of 0.01. Representative photomicrographs depicting Venus fluorescence were acquired at 3 and 7 days post-infection. Scale bars (lower right corner) represent 200 μm. No fluorescence was seen in Vero cells (top row).

FIG. 12 is a set of images of photomicrographs showing rFeMV^{US5} infection of a macrophage cell line. Fcwf-4 cells were infected with rFeMV^{US5}Venus (3) at a multiplicity of infection of 0.01. Representative photomicrographs depicting Venus fluorescence were acquired at 6 days post-infection. Scale bars (lower right corner) represent 200 μm.

### DETAILED DESCRIPTION OF THE INVENTION

Unexpectedly, a method of producing recombinant feline morbillivirus (FeMV) using reverse genetics was developed. The methods of the present invention provide a complete sequence and provide an unaltered sequence. Previously, cell passage or isolation had to be used to obtain FeMV. Cell passage and isolation are not ideal methods for obtaining FeMV because these methods provide the opportunity for FeMV to adapt (e.g., mutate) e.g. to use alternative receptors. Therefore, reverse genetics provides an unaltered sequence ideal for downstream applications.

Specifically, an aspect of the invention provides a method of producing recombinant FeMV, the method comprising using reverse genetics.

A further aspect of the invention provides a method of producing recombinant FeMV, the method comprising: a. extracting FeMV RNA from an isolated FeMV positive sample; b. sequencing the extracted FeMV RNA; c. aligning the extracted FeMV RNA sequences to each other; d. preparing consensus sequences from the aligned FeMV RNA sequences; e. assembling the consensus sequences; and f. preparing full-length recombinant FeMV based on the assembled consensus sequences using amplicons, synthetic DNA, or a combination thereof.

Another aspect of the invention provides a method of producing recombinant feline morbillivirus (FeMV), the method comprising: a. extracting FeMV RNA from an isolated FeMV positive sample; b. generating cDNAs from the FeMV RNA using primers that specifically hybridize to the FeMV RNA; c. generating cDNA PCR amplicons from the cDNAs using primers that specifically hybridize to the cDNAs to produce cDNA PCR amplicons; d. amplifying genomic and antigenomic termini of the FeMV RNA by rapid amplification of cDNA ends (RACE) using one or more RACE primers to produce RACE PCR amplicons; e. purifying the cDNA PCR amplicons of step (c) and the RACE PCR amplicons of step (d) to produce purified DNAs; f. sequencing the purified DNAs to produce consensus sequences; g. assembling the consensus sequences to produce a full-length FeMV genome; and h. assembling the full-length FeMV genome in a plasmid.

An aspect of the invention provides a method of producing recombinant feline morbillivirus (FeMV), the method comprising: a. extracting FeMV RNA from an isolated FeMV positive sample; b. generating cDNAs from the FeMV RNA using primers that specifically hybridize to the FeMV RNA; c. generating cDNA PCR amplicons from the cDNAs using primers that specifically hybridize to the cDNAs to produce cDNA PCR amplicons; d. amplifying genomic and antigenomic termini of the FeMV RNA by rapid amplification of cDNA ends (RACE) using one or more RACE primers to produce RACE PCR amplicons; e. purifying the cDNA PCR amplicons of step (c) and the RACE PCR amplicons of step (d) to produce purified DNAs; f. sequencing the purified DNAs to produce consensus sequences; g. assembling the consensus sequences to produce a full-length FeMV genome; h. assembling the full-length FeMV genome in a plasmid; i. transfecting cells to express feline CD150 (feCD150) and at least one feline cysteine protease to produce precursor producer cells; j. introducing T7 RNA polymerase into the precursor producer cells by transfection or infection; and k. transfecting the precursor producer cells with FeMV nucleo-(N), phospho-(P) and large (L) proteins and the plasmid of (h) to produce producer cells.

Any suitable feline cysteine protease may be used. For example, in an aspect of the invention the feline cysteine protease is a cathepsin.

Any suitable cells may be used as producer cells. In an aspect of the invention, the producer cells are a feline cell line expressing feCD150 and at least one feline cysteine protease. In another aspect of the invention, the precursor producer cells are Crandell Rees feline kidney cells expressing feline CD150 (CRFK-feCD150). In a further aspect, the producer cells are infected using a virus. In another aspect, the producer cells are infected using Modified Vaccinia virus Ankara (MVA).

In an aspect of the invention, the methods further comprise propagating the producer cells to produce the recombinant FeMV. Any suitable propagation methods may be used.

The amplicons referred to herein, e.g., the cDNA PCR amplicons and RACE PCR amplicons, can be created by one skilled in the art based on the sequences provided herein, and general knowledge.

Any suitable RACE primers may used to produce the RACE PCR amplicons. In an aspect of the invention, a synthetic RACE primer is used, for example long anchored d (T) oligos can be used (e.g., d (T) 4VN). In another aspect of the invention, one or more gene-specific RACE primers can be used.

In an aspect of the invention, the RNA can be A-tailed.

An aspect of the invention provides a method of detecting the presence of FeMV in a sample, the method comprising: a. exposing an isolated test sample to primers that specifically hybridize to FeMV RNA and specifically hybridizing the primers to the FeMV RNA; b. reverse transcribing the FeMV RNA to synthesize FeMV cDNA; c. performing PCR amplification on the FeMV cDNA to produce a PCR amplicon; d. detecting the presence of the PCR amplicon; and e. comparing a presence of the PCR amplicon in the at least one test sample with an absence of PCR amplicon from a negative sample that lacks FeMV RNA, wherein detection of the PCR amplicon is indicative of the presence of one or more FeMV.

In an aspect of the invention, the primers that specifically hybridize to the FeMV RNA comprise, consist essentially of, and/or consist of at least one forward primer with at least 90% identity (e.g., at least 91%, 92%, 93%, 94%, 95%, 96%, 97%, 98%, or 99%) to SEQ ID NOs: 1, 3, 7, 9, 11, 13, 15, 17, 19, 20, 22, 24, 25, 27, 30, and/or 33. In a further aspect of the invention, the primers that specifically hybridize to the FeMV RNA comprise, consist essentially of, and/or consist of at least one forward primer comprising, consisting essentially of, and/or consisting of SEQ ID NOs: 1, 3, 7, 9, 11, 13, 15, 17, 19, 20, 22, 24, 25, 27, 30, and/or 33.

In an aspect of the invention, the primers that specifically hybridize to the FeMV RNA comprise, consist essentially of, and/or consist of at least one reverse primer with at least 90% identity (e.g., at least 91%, 92%, 93%, 94%, 95%, 96%, 97%, 98%, or 99%) to SEQ ID NOs: 1, 2, 4, 5, 8, 12, 14, 16, 18, 6, 23, 26, 28, and/or 31. In a further aspect of the invention, the primers comprise, consist essentially of, and/or consist of at least one reverse primer comprising, consisting essentially of, and/or consisting of SEQ ID NOs: 1, 2, 4, 5, 8, 12, 14, 16, 18, 6, 23, 26, 28, and/or 31.

In an aspect of the invention, the primers that specifically hybridize to the FeMV RNA comprise, consist essentially of, and/or consist of at least one cDNA primer with at least 90% identity (e.g., at least 91%, 92%, 93%, 94%, 95%, 96%, 97%, 98%, or 99%) to SEQ ID NOs: 1, 3, 6, 10, 12, 21, 29, and/or 32. In a further aspect of the invention, the primers that specifically hybridize to the FeMV RNA comprise, consist essentially of, and/or consist of at least one cDNA primer comprising, consisting essentially of, or consisting of SEQ ID NOs: 1, 3, 6, 10, 12, 21, 29, and/or 32.

In an aspect of the invention, the at least one consensus sequence has at least 90% identity (e.g., at least 91%, 92%, 93%, 94%, 95%, 96%, 97%, 98%, or 99%) to SEQ ID NOs: 34 and/or 35. In a further aspect of the invention, the at least one consensus sequence comprises, consists essentially of, and/or consists of SEQ ID NO: 34. In a further aspect of the invention, the at least one consensus sequence comprises, consists essentially of, and/or consists of SEQ ID NO: 35.

In an aspect of the invention, the recombinant FeMV comprises genes N, P/V/C, M, F, H, and L. In a further aspect of the invention, the recombinant FeMV comprises genes N, P/V/C, M, F, H, and L in arrangement 3′-N-P/V/C/-M-F-H-L-5′.

In an aspect of the invention, the recombinant FeMV may comprise an additional transcription unit (ATU). In a further aspect of the invention, the additional ATU encodes a detectable reporter protein. Any suitable detectable reporter protein may be used. In an aspect of the invention, the detectable reporter protein is a luciferase. In a further aspect of the invention, the detectable reporter protein is Gaussia luciferase (Gluc), Renilla luciferase, or firefly luciferase. In another aspect of the invention, the detectable reporter protein is a fluorescent reporter protein. In an aspect of the invention, the detectable reporter protein is EGFP, Venus dTomato, or TagBFP.

The additional ATU may be in any suitable position. For example, in an aspect of the invention, the ATU is located between the H and L genes of the recombinant FeMV. In an alternative aspect of the invention, the ATU is located between the P and M genes of the recombinant FeMV. In another aspect of the invention, the ATU is located 3′ to the N gene of the recombinant FeMV. In another aspect of the invention, the ATU is located 5′ to the N gene of the recombinant FeMV.

The FeMV sample can be from any suitable source. For example, in an aspect of the invention, the recombinant FeMV is from feline tissue or fluid (e.g., feline urine, feline blood, feline thymus, feline lymph nodes, feline urinary tract tissue, or feline respiratory tract tissue). In a further aspect of the invention, the FeMV sample is from feline urine.

The recombinant FeMV can be created using any suitable reverse genetics system. The methods described herein are merely exemplary.

Aspects, including embodiments, of the subject matter described herein may be beneficial alone or in combination, with one or more other aspects or embodiments. Without limiting the foregoing description, certain non-limiting aspects of the disclosure numbered (1)-(28) are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
(1) A method of producing recombinant feline morbillivirus (FeMV), the method comprising using reverse genetics.
(2) The method of aspect 1, the method comprising:
   a. extracting FeMV RNA from an isolated FeMV positive sample;
   b. sequencing the extracted FeMV RNA;
   c. aligning the extracted FeMV RNA sequences to each other;
   d. preparing consensus sequences from the aligned FeMV RNA sequences;
   e. assembling the consensus sequences; and
   f. preparing full-length recombinant FeMV based on the assembled consensus sequences using amplicons, synthetic DNA, or a combination thereof.
(3) A method of producing recombinant feline morbillivirus (FeMV), the method comprising:
   a. extracting FeMV RNA from an isolated FeMV positive sample;
   b. generating cDNAs from the FeMV RNA using primers that specifically hybridize to the FeMV RNA;
   c. generating cDNA PCR amplicons from the cDNAs using primers that specifically hybridize to the cDNAs to produce cDNA PCR amplicons;
   d. amplifying genomic and antigenomic termini of the FeMV RNA by rapid amplification of cDNA ends (RACE) using one or more RACE primers to produce RACE PCR amplicons;
   e. purifying the cDNA PCR amplicons of step (c) and the RACE PCR amplicons of step (d) to produce purified DNA;
   f. sequencing the purified DNA to produce consensus sequences;
   g. assembling the consensus sequences to produce a full-length FeMV genome; and
   h. assembling the full-length FeMV genome in a plasmid.
(4) The method of aspect 3, further comprising:
   i. transfecting cells to express feline CD150 (feCD150) and at least one feline cysteine protease to produce precursor producer cells;
   j. introducing T7 RNA polymerase into the precursor producer cells by transfection or infection; and
   k. transfecting the precursor producer cells with FeMV nucleo-(N), phospho-(P) and large (L) proteins and the plasmid of (h) to produce producer cells.
(5) The method of aspect 4, wherein the precursor producer cells are Crandell Rees feline kidney cells expressing feline CD150 (CRFK-feCD150).
(6) The method of aspect 4 or 5, wherein the producer cells are infected using Modified Vaccinia virus Ankara (MVA).
(7) The method of any one of aspects 4-6, further comprising:
   l. propagating the producer cells to produce the recombinant FeMV.
(8) The method of any one of aspects 3-7, wherein the primers comprise at least one forward primer with at least 90% identity to SEQ ID NOs: 1, 3, 7, 9, 11, 13, 15, 17, 19, 20, 22, 24, 25, 27, 30, and 33.
(9) The method of any one of aspects 3-8, wherein the primers that specifically hybridize to the FeMV RNA comprise at least one forward primer comprising SEQ ID NOs: 1, 3, 7, 9, 11, 13, 15, 17, 19, 20, 22, 24, 25, 27, 30, and 33.
(10) The method of any one of aspects 3-9, wherein the primers that specifically hybridize to the FeMV RNA comprise at least one reverse primer with at least 90% identity to SEQ ID NOs: 1, 2, 4, 5, 8, 12, 14, 16, 18, 6, 23, 26, 28, and 31.
(11) The method of any one of aspects 3-10, wherein the primers that specifically hybridize to the FeMV RNA comprise at least one reverse primer comprising SEQ ID NOs: 1, 2, 4, 5, 8, 12, 14, 16, 18, 6, 23, 26, 28, and 31.
(12) The method of any one of aspects 3-11, wherein the primers that specifically hybridize to the FeMV RNA comprise at least one cDNA primer with at least 90% identity to SEQ ID NOs: 1, 3, 6, 10, 12, 21, 29, and 32.
(13) The method of any one of aspects 3-12, wherein the primers that specifically hybridize to the FeMV RNA comprise at least one cDNA primer comprising SEQ ID NOs: 1, 3, 6, 10, 12, 21, 29, and 32.
(14) The method of any one of aspects 3-13, wherein at least one consensus sequence has at least 90% identity to SEQ ID NOs: 34 and 35.
(15) The method of any one of aspects 3-14, wherein at least one consensus sequence comprises SEQ ID NOs: 34 and 35.
(16) The method of any one of aspects 1-15, wherein the recombinant FeMV comprises genes N, P/V/C, M, F, H, and L.
(17) The method of any one of aspects 1-16, wherein the recombinant FeMV comprises genes N, P/V/C, M, F, H, and L in arrangement 3′-N-P/V/C/-M-F-H-L-5′.
(18) The method of any one of aspects 1-17, wherein the recombinant FeMV further comprises an additional transcription unit (ATU) encoding a detectable reporter protein.
(19) The method of aspect 18, wherein the ATU is located between the H and L genes of the recombinant FeMV.
(20) The method of aspect 18, wherein the ATU is located between the P and M genes of the recombinant FeMV.
(21) The method of aspect 18, wherein the ATU is located 3′ to the N gene of the recombinant FeMV.
(22) The method of any one of aspects 2-21, wherein the FeMV sample is from feline urine.
(23) The method of any one of aspects 2-22, wherein the recombinant FeMV is from feline urine, feline blood, feline thymus, feline lymph nodes, feline urinary tract tissue, or feline respiratory tract tissue.
(24) A method of detecting the presence of FeMV in a sample, the method comprising:
a. exposing an isolated test sample to primers that specifically hybridize to FeMV RNA and specifically hybridizing the primers to the FeMV RNA;
b. reverse transcribing the FeMV RNA to synthesize FeMV cDNA;
c. performing PCR amplification on the FeMV cDNA to produce a PCR amplicon;
d detecting the presence of the PCR amplicon; and
e. comparing a presence of the PCR amplicon in the at least one test sample with an absence of PCR amplicon from a negative sample that lacks FeMV RNA, wherein detection of the PCR amplicon is indicative of the presence of one or more FeMV.
(25) The method of aspect 24, wherein the primers that specifically hybridize to the FeMV RNA comprise at least one forward primer with at least 90% identity to SEQ ID NOs: 1, 3, 7, 9, 11, 13, 15, 17, 19, 20, 22, 24, 25, 27, 30, and 33.
(26) The method of aspect 24 or 25, wherein the primers that specifically hybridize to the FeMV RNA comprise at least one forward primer comprising SEQ ID NOs: 1, 3, 7, 9, 11, 13, 15, 17, 19, 20, 22, 24, 25, 27, 30, and 33.
(27) The method of any one of aspects 24-26, wherein the primers that specifically hybridize to the FeMV RNA comprise at least one reverse primer with at least 90% identity to SEQ ID NOs: 1, 2, 4, 5, 8, 12, 14, 16, 18, 6, 23, 26, 28, and 31.
(28) The method of any one of aspects 24-27, wherein the primers that specifically hybridize to the FeMV RNA comprise at least one reverse primer comprising SEQ ID NOs: 1, 2, 4, 5, 8, 12, 14, 16, 18, 6, 23, 26, 28, and 31.

The following example further illustrates the invention but, of course, should not be construed as in any way limiting its scope.

### Example 1

This example demonstrates the production of FeMV using reverse genetics.

### Cells

The Crandell-Rees feline kidney (CRFK) epithelial cell line and the feline macrophage cell line Fcwf-4 were obtained from ATCC (Virginia, USA) and grown in Eagle's minimum essential medium (ATCC), supplemented with 10% (vol/vol) fetal bovine serum (Thermo Fisher Scientific). The CRFK-feCD150 and CRFK-hCD150 derivative cells were grown in the same medium with periodic passage in the presence of Puromycin (500 μg/ml) to maintain expression of CD150. Hep-2 cells were grown in Opti-MEM I supplemented with 3% (vol/vol) fetal bovine serum (both Thermo Fisher Scientific). 293T cells were purchased from ATCC (Virginia, USA) and grown in Advanced minimal essential medium supplemented with 10% (vol/vol) fetal bovine serum (both Thermo Fisher Scientific).

### Generation of CRFK-feCD150 and CRFK-hCD150 Stable Cell Lines

DNA strings encoding feline CD150 (feCD150; accession number NM 001278826) or human CD150 (hCD150; accession number NM 003037.5) were synthetically generated (GeneArt Gene Synthesis; Thermo Fisher Scientific) and cloned into a lentiviral expression vector which also encoded puromycin resistance to generate pHAGE^{puro}-feCD150 and pHAGE^{puro}-hCD150. pHAGE^{puro}-feCD150 or pHAGE^{puro}-hCD150 and helper plasmids expressing HIV-gag and pol, and VSV-G were co-transfected into 293T cells using lipofectamine 2000 (Thermo Fisher Scientific). Lentivirus-containing supernatants were collected every 12 hours for two consecutive days starting at 48 hours post-transfection (h.p.t.). The supernatants were pooled and filtered through a sterile 0.45 μm filter (Millipore) to remove any residual cells. The lentiviral particles were concentrated by centrifugation through 20% sucrose at 28,000 g for 2 hours at 4° C. The pellet was resuspended in 200 μl phosphate buffered saline (Thermo Fisher Scientific) and 20 μl were used to transduce 5×10⁵ CRFK cells seeded in a 6-well culture plate in the presence of polybrene (5 μg/ml; Sigma Aldrich). Cells were then selected using puromycin (5 μg/ml; Thermo Fisher Scientific) two days after the transduction.

### Plasmids

The F and H glycoprotein sequences of FeMV^{US1} (Sharp, et al., Emerg. Infect. Dis., 22 (2016)), FeMV^{US2} (accession numbers ON783815 and ON783816) and FeMV^{US5}, and the N, P, and L protein sequences of FeMV^{US5} were generated by PCR and cloned into the eukaryotic expression vector pCG (Cathomen, et al., Virology, 214:628-632 (1995)) using unique Asc I and Afe I restriction sites to generate pCG-FeMV^{US1}F, pCG-FeMV^{US1}H, pCG-FeMV^{US5}P and pCG-FeMV^{US5}L. pCG-FeMV^{US5}F was modified by insertion of a PCR generated insert containing an AU1 epitope tag at the C-terminus of FeMV^{US5}F between unique EcoR V and Afe I restriction sites to generate pCG-FeMV^{US5}F_{AU1}. A synthetically generated gene string (GeneArt Gene Synthesis) containing a polybasic cleavage signal at the original monobasic cleavage site in FeMV^{US5}F was used to modify pCG-FeMV^{US5}F_{AU1} by cloning between unique Asc I and BsrG I restriction sites to generate pCG-FeMV^{US5}F_{PB-AU1}. The F and H glycoprotein sequences of MV^{KS} (Lemon, et al., PLOS Pathog., 7: e1001263 (2011)) and CDV^{RI} (Tilston-Lunel, et al., mSphere, 6: e0053721 (2021)), and the F and G glycoprotein sequences of NiV (Harcourt, et al., Virology, 271:334-349 (2000)) were generated by PCR and cloned into pCG using unique Asc I and Spe I restriction sites to generate pCG-MV^{KS}F, pCG-MV^{KS}H, pCG-CDV^{RI}F, pCG-CDV^{RI}H, pCG-NiV^{MA}F and pCG-NiV^{MA}G. Sequences for NrlucEGFP and rlucEGFPC were amplified from plasmid templates (Kelly, et al., Viruses, 11 (2019); Ishikawa, et al., Protein Eng. Des. Sel., 25:813-820 (2012)) by PCR and cloned into pCG using unique Mlu I and Pst I restriction sites to generate the pCG-NrlucEGFP and pCG-rlucEGFPC plasmids used in the bimolecular fluorescence complementation assay. The sequence for FeMV^{US5}DIGluc was generated synthetically (GeneArt Gene Synthesis) and cloned into a modified pBluescript plasmid (Sidhu, et al., Virology, 208:800-807 (1995)) using unique Nar I and Not I restriction sites to generate p(−)FeMV^{US5}DIGluc. This contains a Gaussia luciferase (Gluc) open reading frame (ORF), flanked by the FeMV^{US5} 3′ and 5′ non-coding termini and surrounded by a T7 RNA polymerase promoter downstream, and by a hepatitis delta virus ribozyme and T7 terminator sequences upstream. A synthetically generated genestring (GeneArt Gene Synthesis) was used to modify p(−)FeMV^{US5}DIGluc by removing an extra stop codon after the Gluc ORF and was cloned using unique Nco I and BsrG I restriction sites to generate p(−)FeMV^{US5}DIGluc+3. Both plasmids produce negative sense minigenome transcripts upon T7 RNA polymerase transcription.

### Inhibitors

The cysteine protease inhibitor, E64d (Sigma-Aldrich) was used at a concentration of 20 μM. The cell permeable cathepsin B/L inhibitor, CA-074ME (Calbiochem) was used at a concentration of 10 μM. The furin inhibitor, furin inhibitor I (Calbiochem) was used at a concentration of 50 μM. All inhibitors were dissolved in sterile dimethyl sulfoxide (DMSO). For fusion assays, inhibitors were added after the transfection of glycoprotein-expressing plasmids. For virus assays inhibitors were added with the virus inoculum. In both cases, controls containing the same volume of DMSO were included and fresh inhibitor/DMSO was supplied with media changes.

### Bimolecular Fluorescence Complementation Assay

This assay was based on the previously published self-associating split GFP (Ishikawa, et al., Protein Eng. Des. Sel., 25:813-820 (2012); Thakur, et al., J. Gen. Virol., 102 (2021)). Subconfluent CRFK cells in 6-well trays were transfected with 1 μg each of pCG-NrlucEGFP and plasmids encoding homologous pairs of glycoproteins. Separate wells of CRFK cells (for controls) or CRFK-feCD150 or CRFK-hCD150 cells were transfected with pCG-rlucEGFPC. At 18 h.p.t., all cells were trypsinized and mixed in appropriate combinations before re-seeding in 6-well trays and further incubation. Cells were observed using a DMI3000B inverted microscope and images were acquired using a DFC345 FX camera and LAS software (all Leica Microsystems) when sufficient green fluorescence was detected (at 48-72 h.p.t). At this point the growth medium was removed, the cells washed once with PBS (1 ml) and 1× lysis buffer (500 μl; Renilla Luciferase assay system, Promega) was added before scraping the cells into the supernatant. The cell lysates were collected into 1.5 ml microcentrifuge tubes and supernatants were collected by centrifugation at 6,000 r.p.m. for 1 minute. The supernatants were assayed by addition of 1 μl (diluted in 49 μl of lysis buffer) to 50 μl of rLuc substrate (Renilla Luciferase assay system, Promega) followed immediately by light quantification using a LUMISTAR Omega luminometer (BMG Labtech). The resulting luciferase activity is expressed as relative light units (R.L.U.).

For assays with virus, separate populations of CRFK-feCD150 cells were transfected (Lipofectamine 2000, Life Technologies) with pCG-NrlucEGFP or pCG-rlucEGFPC (1 μg plasmid per 10⁶ cells). 24 hours later cells transfected with pCG-NrlucEGFP were infected with rFeMV^{US5}Venus (6) at a multiplicity of infection (M.O.I.) of 0.1. Infection was performed in the presence of inhibitors. After 24 hours these cells were overlaid (in the presence of inhibitor) with the population of CRFK-feCD150 cells that had been transfected with pCG-rlucEGFPC. After incubation with inhibitors for 2 days, monolayers were assessed for luciferase activity as described above for glycoprotein assays.

### Sample Preparation, PAGE and Western Blotting

CRFK cells were transfected with pCG-FeMV^{US5}F, pCG-FeMV^{US5}F_{AU1}, or pCG-FeMV^{US5}F_{PB-AU1} in the absence or presence of the pan-cysteine protease inhibitor E64d. At 2 d.p.t. cell lysates were prepared. Medium was removed and the monolayers were rinsed twice with 1 ml cold D-PBS. Cold 1× RIPA buffer (Boston bioproducts; 200 μl) containing 1× HALT protease inhibitors (Thermo Fisher Scientific) was added to the monolayers and incubated on ice for 15 minutes. Monolayers were scraped into the buffer and transferred to cold 1.5 ml tubes. Lysates were incubated on ice for 30 minutes with intermittent vortexing before being centrifuged at 14,000 g for 15 minutes at 4° C. to pellet nuclei. The cleared supernatants were used to prepare samples for polyacrylamide gel electrophoresis (PAGE) by adding appropriate volumes of 4× NUPAGE™ LDS sample loading buffer and ×10 NUPAGE™ reducing agent (both Thermo Fisher Scientific Scientific). PAGE samples were heated to 70° C. for 10 minutes before separation of proteins on a 10% NUPAGE™ bis-TRIS polyacrylamide gel using the Xcell SureLock Mini-Cell system (Thermo Fisher Scientific Scientific) according to manufacturer's instructions. An aliquot of SeeBlue Plus2 Protein Standard was included on each gel to allow estimation of protein sizes. Proteins were transferred to nitrocellulose using an iBlot (standard 7 minutes at 20V transfer protocol; Thermo Fisher Scientific) according to manufacturer's instructions. Blots were blocked for 1 hour in ODYSSEY™ blocking buffer (PBS, Licor). Blots were incubated with primary antibodies (rabbit anti-AU1, 1:1000, Novus biologicals and mouse anti-β-actin, 1:5000, Abcam, diluted in 50:50 ODYSSEY™ blocking buffer: PBS/0.2% (vol/vol) TWEEN™-20) overnight at 4° C.

Primary antibodies were removed and blots were washed 3 times for 15 minutes with excess PBS. Blots were incubated with secondary antibodies (goat anti-rabbit-680, 1:10000 and goat anti-mouse-800, 1:10000, both Licor, diluted in 50:50 ODYSSEY™ blocking buffer: PBS/0.2% (vol/vol) TWEEN™-20) with rocking for 1 hour at room temperature. Secondary antibodies were removed and blots were washed 3 times for 15 minutes with excess PBS before imaging using an ODYSSEY™ CLx (Licor) according to manufacturer's instructions.

### Minigenome Assays

Hep-2 cells were grown to 80% confluency in 24 well trays, rinsed with Opti-MEM I (1 ml; Thermo Fisher Scientific) and infected with MVA-T7 at an M.O.I. of 1 for 45 minutes. Lipofectamine 2000 (Thermo Fisher Scientific) was diluted with Opti-MEM I according to manufacturer's instructions and incubated at room temperature for 10 minutes. A DNA mixture containing pCG-FeMV^{US5}N, pCG-FeMV^{US5}P, and pCG-FeMV^{US5}L eukaryotic expression plasmids and either p(−)FeMV^{US5}Gluc or p(−)FeMV^{US5}Gluc+3 was added and liposome-DNA complexes were formed by incubation for 20 minutes at room temperature. The MVA-T7 inoculum was removed and the complexes spotted onto the Hep-2 cell monolayers. Opti-MEM I (1 ml) was added to each well. After 18 hours incubation at 37° C. the complexes were replaced with Opti-MEM I (1 ml) containing 3% (vol/vol) fetal bovine serum (Thermo Fisher Scientific). Supernatant samples were collected at 48 h.p.t. and were assayed by addition of 100 ng native coelenterazine substrate (Nanolight Technologies) in D-PBS (Thermo Fisher Scientific) followed immediately by light quantification using a LUMISTAR Omega luminometer (BMG Labtech). The resulting Gaussia luciferase activity is expressed as relative light units (R.L.U.).

### FeMV^{US5} Sequence Determination

A urine sample was collected by cystocentesis from a male, neutered, healthy, pet, domestic shorthair cat. All RNA extraction, cDNA synthesis, and PCR was performed in a clean room, using dedicated pipettes, kits, enzymes, primers, and plasticware. cDNA synthesis and PCRs were set up using different pipettes. A reverse-transcriptase-negative control was included to demonstrate that amplicons were not attributable to contamination. No tube that might contain an FeMV amplicon was ever opened in the clean room. All DNA gel electrophoresis was performed in a separate laboratory on a different floor.

Total RNA was extracted using a Viral RNA Minikit (Qiagen) and cDNA was prepared using SuperScript III reverse transcriptase (Thermo Fisher Scientific) priming with random hexamers. Screening (Sharp, et al., Emerg. Infect. Dis., 22 (2016)) identified the sample as positive for FeMV RNA. Primers (Table 3) were used to generate additional cDNAs from the extracted total RNA and generate PCR amplicons which were either purified using a QIAQUICK™ PCR purification kit (Qiagen) or were gel-extracted and purified using a QIAQUICK™ gel extraction kit (Qiagen) before sequencing (Genewiz) with the same primers used to amplify the target region. Initially primers (Table 1, Asia and 776U designations) were designed using alignments of published FeMV sequences to identify highly conserved regions. Once FeMV^{US5} sequence was available from these amplicons, FeMV^{US5} specific primers (Table 3, U122 and US5 designations) were designed for cDNA synthesis, PCR, and sequencing. Rapid amplification of cDNA ends was used to generate amplicons containing leader and trailer sequences as previously described (Rennick, et al., J. Gen. Virol., 101:1056-1068 (2020)); these were sequenced to determine the authentic genomic termini of FeMV^{US5}. Sequences were aligned in DNASTAR SeqMan Pro software (Lasergene) and contigs were generated corresponding to the consensus sequence. DNAstar SeqBuilder software (Lasergene) was used to assemble and annotate the complete genome sequence. The complete FeMV^{US5} sequence is available with accession number MN604235

TABLE 1
Primers used for the reverse transcription and amplification of the complete genome of FeMV^{US5} from total RNA

| Amplicon | CDNA | cDNA primer sequence | Forward | Sequence | Reverse | | |
|---|---|---|---|---|---|---|---|
| (bp) | synthesis | (5′ to 3′) | primer | (5′ to 3′) | primer | Sequence (5′ to 3′) | Kit* |
| Leader | priAdaptor- | GACTCGAGTCGACAT | priAdaptor- | GACTCGAGTCGA | priFeMV^{U122} | CATCAGGATCA | P |
| (RACE) | dT17 | CGATTTTTTTTTTTTTT | dT17 | CATCGATTTTTT | 376- | TCAGCGATTC | |
| (431) | | TTT (SEQ ID NO: 1) | | TTTTTTTTTTT | | (SEQ ID NO: 2) | |
| | | | | (SEQ ID NO: 1) | | | |
| Leader-N | priFeMV^{Asia} | ACCAGACAAAGATGT | priFeMV^{Asia} | ACCAGACAAAG | priFeMV^{U122} | CCTATCAGTGA | P |
| (1394) | 20+ | CTGTG (SEQ ID NO: 3) | 20+ | ATGTCTGTG | 1371- | TGTTGTCCTTAG | |
| | | | | (SEQ ID NO: 3) | | C (SEQ ID NO: 4) | |
| Leader-N | priFeMV^{Asia} | ACCAGACAAAGATGT | priFeMV^{Asia} | ACCAGACAAAG | priFeMV^{U122} | CAATTCTATATG | P |
| (1485) | 20+ | CTGTG (SEQ ID NO: 3) | 20+ | ATGTCTGTG | 1464- | AGTCACCTGAG | |
| | | | | (SEQ ID NO: 3) | | (SEQ ID NO: 5) | |
| Leader-N | priFeMV^{U122} | AGAGTAGTCCTTCCGC | priFeMV^{776U} | TGTGACCTATTC | priFeMV^{U122} | GAAATGTTGTT | P |
| (1349) | 7132- | TTATAG | 38+ | TAACGACAAG | 1345- | GGGTTTGCTC | |
| | | (SEQ ID NO: 6) | | (SEQ ID NO: 7) | | (SEQ ID NO: 8) | |
| N | priFeMV^{Asia} | ACCAGACAAAGATGT | priFeMV^{Asia} | CGTCAGGTTCAG | priFeMV^{U122} | CCTATCAGTGA | P |
| (1226) | 20+ | CTGTG (SEQ ID NO: 3) | 187+ | GTGGTAC (SEQ | 1371- | TGTTGTCCTTAG | |
| | | | | ID NO: 9) | | C (SEQ ID NO: 4) | |
| N-H | priFeMV^{U122} | GGTTTAGTGCAGGGT | priFeMV^{U122} | AGGGTCTTATCC | priFeMV^{U122} | CTTGCTAAAAT | P |
| (5926) | 1073+ | CTTATC (SEQ ID NO: | 1109+ | TCTGCTATGGAG | 6994- | ACCGGTTTGAA | |
| | | 10) | | (SEQ ID NO: 11) | | TC (SEQ ID NO: | |
| | | | | | | 12) | |
| N-F | priFcMV^{U122} | AGGGTCTTATCCTCTG | priFcMV^{U122} | TCCAATTCTGCT | priFcMV^{U122} | GAGGGAGGTAT | P |
| (4995) | 1109+/ | CTATGGAG (SEQ ID | 1569+ | ATGGAAGAG | 6526- | GTAGAGGGGTT | |
| | priFeMV^{U122} | NO: | | (SEQ ID NO: 13) | | C (SEQ ID NO: 14) | |
| | 6994- | 11)/CTTGCTAAAATAC | | | | | |
| | | CGGTTTGAATC (SEQ | | | | | |
| | | ID NO: 12) | | | | | |
| P-F | priFeMV^{U122} | AGGGTCTTATCCTCTG | priFeMV^{U122} | TGCCAGGGAAAT | priFeMV^{U122} | ACGTGAATTTA | P |
| (4536) | 1109+/ | CTATGGAG (SEQ ID | 1894+ | TTACGAATC | 6393- | AAGAATCCAAC | |
| | priFeMV^{U122} | NO: | | (SEQ ID NO: 15) | | (SEQ ID NO: 16) | |
| | 6994- | 11)/CTTGCTAAAATAC | | | | | |
| | | CGGTTTGAATC (SEQ | | | | | |
| | | ID NO: 12) | | | | | |
| M-F | priFeMV^{U122} | GGTTTAGTGCAGGGT | priFeMV^{U122} | AGGTATTGTCAC | priFeMV^{U122} | CTACCTGTGCA | P |
| (440) | 1073+ | CTTATC (SEQ ID NO: | 4557+ | TCCAGCTTAC | 4997- | AATGTAATATC | |
| | | 10) | | (SEQ ID NO: 17) | | TG (SEQ ID NO: | |
| | | | | | | 18) | |
| F-H | random | — | priFeMV^{Asia} | AGGACCTAGAA | priFeMV^{U122} | CTTGCTAAAAT | P |
| (795) | hexamers | | 6241+ | AATTACCAG | 6994- | ACCGGTTTGAA | |
| | | | | (SEQ ID NO: 19) | | TC (SEQ ID NO: | |
| | | | | | | 12) | |
| F-H | random | — | priFeMV^{U122} | GCAATACTATCC | priFeMV^{U122} | AGAGTAGTCCT | P |
| (351) | hexamers | | 6826+ | TATACACATG | 7132- | TCCGCTTATAG | |
| | | | | (SEQ ID NO: 20) | | (SEQ ID NO: 6) | |
| F-L | priFeMVUS5 | ATCATGCATCCGCTGT | priFeMV^{M252A} | AAAACTTAGGA | priFeMV^{M252A} | CCACAAAATGA | Q |
| (3300) | 10493R | AATTAG (SEQ ID NO: | 6946+ | ATCCATGTT | 10205- | CCTCCAGTA | |
| | | 21) | | (SEQ ID NO: 22) | | (SEQ ID NO: 23) | |
| L | random | — | priFcMV^{U122} | CACCATTGCCTT | priFcMV^{M252A} | CCACAAAATGA | P |
| (419) | hexamers | | 9821+ | TCAGAGTTG | 10205- | CCTCCAGTA | |
| | | | | (SEQ ID NO: 24) | | (SEQ ID NO: 23) | |
| L | random | — | RMH F2 | GCCATATTTTGT | RMH R | CTCATTTTGTAI | T |
| (493) | hexamers | | | GGAATAATHATH | | GTCATYTTNGC | |
| | | | | AAYGG (SEQ ID | | RAA (SEQ ID NO: | |
| | | | | NO: 25) | | 26) | |
| L | random | — | priFeMV^{U122} | GCTCTCGAAGAT | priFeMV^{U122} | CAAGATTCCAT | P |
| (3546) | hexamers | | 10393+ | TAGTCAATG | 13984- | GTTAGACCTAA | |
| | | | | (SEQ ID NO: 27) | | TG (SEQ ID NO: | |
| | | | | | | 28) | |
| L-Trailer | priFeMV^{US5} | AGACTATATGAGAGA | priFeMV^{M252A} | GGATGCTTATTT | priFeMV^{M252A} | CCAGACAAAGA | Q |
| (2380) | 10132F | TTGAACTC (SEQ ID | 13689+ | ATCTGATC (SEQ | 16028- | AAGCTATAGG | |
| | | NO: 29) | | ID NO: 30) | | (SEQ ID NO: 31) | |
| Trailer | priFeMV^{U122} | GTGTATTGAGTTGGTA | priFeMV^{U122} | GATAGCGTAATA | priAdaptor- | GACTCGAGTCG | P |
| (RACE) | 15515+ | ATATTC (SEQ ID NO: | 15758+ | TAACAGGTG | dT17 | ACATCGATTTTT | |
| (338) | | 32) | | (SEQ ID NO: 33) | | TTTTTTTTTTTT | |
| | | | | | | (SEQ ID NO: 1) | |

*P, Phusion High-Fidelity DNA-Dependent DNA Polymerase (New England Biolabs, Ipswich, MA, USA); T, Taq DNA-Dependent DNA Polymerase (Thermo Fisher Scientific, Grand Island, NY, USA); Q, Q5 High-Fidelity DNA-Dependent DNA Polymerase (New England Biolabs).

### Generation of Full-Length Clones and Recombinant Virus

Large amplicons from the FeMV^{US5} sequence determination were modified to incorporate an A overhang using Taq DNA Polymerase (Thermo Fisher Scientific) and subcloned using the TOPO TA Cloning Kit for Subcloning (Thermo Fisher Scientific). Clones were sequenced (Genewiz) to identify those which matched the consensus FeMV^{US5} sequence. A cloning strategy was devised based on available cloned DNA and unique restriction sites. A subclone was generated containing some viral sequences and the restriction sites necessary for the cloning strategy in a modified pBluescript vector (Lemon, et al., Journal of Virology, 81:8293-8302 (2007)). The full-length pFeMV^{US5} plasmid was generated by stepwise modifications of this subclone by insertion of sequences from the TOPO-cloned fragments using the appropriate subclone restriction sites.

To make pFeMV^{US5}Venus (3) and pFeMV^{US5}Venus (6) one of the TOPO-cloned fragments used in the generation pFeMV^{US5} was modified with synthetic DNA (GeneArt Gene Synthesis) to insert an additional transcription unit (ATU) encoding Venus fluorescent protein between the P and M genes (pFeMV^{US5}Venus (3)) or H and L genes (pFeMV^{US5}Venus (6)). Appropriate restriction sites were used to switch the modified TOPO-cloned fragment containing the ATU into pFeMV^{US5}.

CRFK feCD150 cells were infected with recombinant vaccinia virus MVA-T7 for 1 h at 37° C. Inoculum was aspirated, and cells were transfected (Lipofectamine 2000, Life Technologies) with pCG-FeMV^{US5}N, pCG-FeMV^{US5}P, PCG-FeMV^{US5}L, and pFeMV^{US5}Venus (6) or pFeMV^{US5}Venus (3). After 18 h the transfection mix was removed and replaced with growth medium Advanced MEM (ATCC) containing 10% (vol/vol) fetal bovine serum (Life Technologies, USA). Cells were incubated for up to 5-7 days at 37° C. with 5% (vol/vol) CO2. The presence of virus was confirmed by cytopathic effect observed by phase-contrast microscopy and fluorescent microscopy. Virus stocks were prepared by trypsinizing cells in a virus positive well and expanding to a T75 flask; when cytopathic effect was maximal monolayers were subjected to one freeze-thaw cycle and debris was removed by centrifugation at 3,000 RPM for 10 minutes at 4° C. The cleared supernatant (virus stock) was aliquoted and titrated in CRFK-feCD150 cells; calculated quantities, expressed in TCID₅₀ units (Reed, et al., Am. J. Hyg., 27:493-497 (1938)) were used to calculate M.O.I.s for infections. Large volumes of virus stock were prepared in the presence of ruxolitinib (0.5-2.0 mM/ml to enhance the virus production (Stewart, et al., PLOS One, 9: e112014 (2014)). The virus stock was then subjected to high-speed centrifugation through 20% (w/vol) sucrose (Sigma) to generate purified virus stock for animal infections. Purified stocks were titrated as above.

### Multistep Growth Analysis

CRFK-feCD150 cells in suspension were infected with rFeMV^{US5}Venus (6) or rFeMV^{US5}Venus (3) in triplicate at an M.O.I. of 0.1 for 4 hours at 37° C. The cells were spun out of the inoculum at 700 g for 5 minutes, the pellet was resuspended, and the cell suspension was divided into aliquots in 36-mm-diameter wells (5×10⁵ cells/well). At each indicated time point the cells and medium were combined into a tube and subjected to one freeze-thaw cycle to release total virus. Virus present in the sample for each time point was determined by endpoint titration in CRFK-feCD150 cells, and quantities are expressed in TCID₅₀ units (Reed, et al., Am. J. Hyg., 27:493-497 (1938)).

### Animal Study Design

Animal experiments were conducted in compliance with all applicable U.S. Federal policies and regulations and AAALAC International standards for the humane care and use of animals. Protocols were approved by the Boston University institutional animal care and use committee. Animals were housed in groups and cages contained appropriate sources of environmental enrichment. Animals were observed several times per day and all procedures were performed under light anesthesia using ketamine, medetomidine and butorphanol followed by atipamezole reversal after handling. To determine the peak of infection, three 16-17 week old, male, domestic shorthair cats were infected with rFeMV^{US5}Venus (6) and rFeMV^{US5}Venus (3) (10⁶ TCID₅₀ each intratracheal and 2×10⁵ TCID₅₀ each intranasal). Twenty days prior to infection cats were implanted (intraperitoneal) with data loggers programed to record core temperature every 10 seconds. Surgery sites were examined frequently and were fully healed prior to infection. Samples were collected from all living animals at various time points: small blood samples were collected on 2, 4, 6, 8, 10, 12, 14, and 21 d.p.i., urine samples were collected on 6, 12, and 21 d.p.i., and throat and nose swabs were collected on 2, 6, 12, 17, and 21 d.p.i. One animal was euthanized on 7, 14, and 28 d.p.i. and full necropsies were performed. To further examine and confirm the peak of infection, three 16-17 week old, male, domestic shorthair cats were infected with rFeMV^{US5}Venus (6) and rFeMV^{US5}Venus (3) (10⁶ TCID₅₀ each intratracheal and 2×10⁵ TCID₅₀ each intranasal). Twenty days prior to infection cats were implanted (subcutaneous) with data loggers programed to record temperature every 5 seconds. Small blood samples were collected on 2, 5, 6, and 7 d.p.i. All animals were euthanized on 7 d.p.i. and full necropsies were performed.

### Samples and Assays

Small blood samples were collected in Vacuette tubes containing EDTA as anticoagulant. Before further processing 50 μl were analyzed on a VetScan HM5 (Abaxis), using cat specific parameters, according to manufacturer's instructions. Red blood cells (RBC) were lysed in the remaining sample using ×1 multi-species RBC lysis buffer (eBioscience) and the remaining white blood cells (WBC) were collected by centrifugation (350 g for 10 minutes), washed 3 times with D-PBS (Thermo Fisher Scientific) and resuspended in an appropriate volume of D-PBS based on pellet size. The WBC were used directly for flow analysis using a LSRII flow cytometer (BD biosciences). Venus fluorescence was detected by excitation with SORP Blue (488 nm) laser and detection using the Octagon detector array and FITC parameter (505 LP mirror and 530/30 BP filter). The WBCs were also used for virus isolations by co-culture with CRFK-feCD150 cells, and screening for the development of Venus fluorescent protein. Urine samples were collected by cystocentesis; a 22-24G (1-1.5 in) needle was used to enter the bladder percutaneously to withdraw a sample of up to 5 ml (max) of urine. Urine (1 ml) was directly used to inoculate confluent monolayers of CRFK-feCD150 in 6-well trays. The inoculum was allowed to adsorb for 2 hours at 37° C. before removal. Monolayers were washed twice before addition of 2 ml CRFK medium. Monolayers were screened for the development of Venus fluorescent protein. Nose and throat swabs were collected into 1 ml virus transport medium. The medium was used directly for virus isolation by titration on CRFK-feCD150 cells in 96-well trays, and screening for the development of Venus fluorescent protein.

At necropsy tissues were collected directly into formalin for fixation and subsequent pathological processing and assessment. Lymph nodes were also collected into D-PBS for subsequent preparation of single cell suspensions. Fatty tissue was removed from the lymph nodes which were dissected into small pieces and added to GENTLEMACS™ dissociation C tubes (Miltenyi Biotec) containing Advanced RPMI medium supplemented with 10% (w/vol) fetal bovine serum, 1% (vol/vol) Glutamax and ×1 Antibiotic-Antimycotic (all Thermo Fisher Scientific). Samples were dissociated using a GENTLEMACS Dissociator (Miltenyi Biotec) set to the m_spleen_C preset parameter and transferred through 100 μm FALCON™ Cell Strainers into 15 ml centrifuge tubes (Thermo Fisher Scientific). The dissociated cells were collected by centrifugation (350 g for 10 minutes), washed once with D-PBS (Thermo Fisher Scientific) and resuspended in an appropriate volume of D-PBS based on pellet size. The dissociated cells were used directly for flow analysis as above for WBC. Bronchoalveolar lavage (BAL) samples were collected by insertion of an appropriately sized nasogastric tube into a primary mainstem bronchus, instillation of 10-15 ml of sterile saline using an attached syringe followed by rapid retraction of as much saline as possible. BAL cells were collected by centrifugation (350 g for 10 minutes), washed once with D-PBS (Thermo Fisher Scientific) and resuspended in an appropriate volume of D-PBS based on pellet size. The cells were used directly for flow analysis and virus isolation as above for WBC.

### Macroscopic Detection of Venus Fluorescence

To examine fluorescence in the body cavity the area was illuminated with a custom-made lamp containing 6 LEDs with peak emission 490-495 nm and viewed through amber glasses which transmitted green light. Images were acquired using an iPhone 8 (Apple) and amber filter.

### Chromogenic Immunohistochemistry (IHC)

Tissues were stained immunohistochemically to detect the presence of Venus protein (surrogate of viral infection) in affected tissues. IHC was performed by an automated Ventana BenchMark ULTRA platform. 5 μm sections were deparaffinized in a xylene bath and rehydrated through graded ethanol solutions. Antigen retrieval was completed for 36 minutes at 95° C. using ULTRA CC1 (Roche). 100 μl of rabbit polyclonal anti-Green fluorescent protein diluted at 1:400 (A11122; Invitrogen) was incubated on each slide at 37° C. for 32 minutes. Venus is a red-shifted variant of GFP, with only 9 amino acid changes to that of GFP; furthermore, GFP antibodies are pan-GFP variant. 100 μl UV Red UNIV MULT (Roche) was dispensed onto each slide and incubated for 12 minutes at 36° C. 100 μl of UV Red Enhancer (Roche) was dispensed onto each slide and incubated for 4 minutes at 36° C. 100 μl each of UV Fast Red A and UV Red Napthol (Roche) were dispensed onto each slide and incubated for 8 minutes at 36° C. 100 μl of UF Fast Red B (Roche) was dispensed onto each slide and incubated for 8 minutes at 36° C. 100 μl Hematoxylin II (Roche) was dispensed onto each slide and incubated for 8 minutes. 100 μl Bluing Reagent (Roche) was dispensed onto each slide and incubated for 8 minutes. All washes in-between steps were completed with ready-to-use reaction buffer (Roche). Slides were removed from the autostainer and rinsed with water and dishwashing detergent and dehydrated through graded alcohols and xylene. Slides were cover slipped using an automated cover slipper and cover slipping film. A lung section from a cat determined to no longer have systemic infection was used simultaneously as a negative control for EGFP.

### Chromogenic In Situ Hybridization (ISH)

ISH targeting viral Venus mRNA was performed on formalin-fixed, paraffin-embedded (FFPE) tissues using the RNAscope 2.5 high definition (HD) RED kit (Advanced Cell Diagnostics) according to the manufacturer's instructions. Briefly, 14 ZZ probe pairs targeting the Venus gene were designed and synthesized by Advanced Cell Diagnostics (493891, Advanced Cell Diagnostics). After deparaffinization with xylene, a series of ethanol washes and peroxidase blocking, sections were heated in Antigen Retrieval Buffer (Advanced Cell Diagnostics) and then digested by proteinase plus (Advanced Cell Diagnostics). Sections were exposed to ISH target probe and incubated at 40° C. in a hybridization oven (HybEZ, Advanced Cell Diagnostics) for 2 h. After rinsing, the ISH signal was amplified using company-provided pre-amplifier and amplifier conjugated to alkaline phosphatase (AP) and incubated with a red substrate-chromogen solution for 10 minutes at room temperature. A Felis catus specific probe targeting the PPIB gene (455011, Advanced Cell Diagnostics) and Bacillus subtilis probe targeting the DApB gene (310043, Advanced Cell Diagnostics) were utilized as positive and negative controls respectively. Sections were then counterstained with hematoxylin, air-dried, and cover slipped.

Multiplex Fluorescent Immunohistochemistry (mIHC)

Tissue sections (5 μm) were baked at 60° C. for an hour and deparaffinized with xylene and a graded series of ethanol. Antigen retrieval was conducted using a Decloacking chamber (Biocare medical) at 90° C. for 15 minutes in AR6 buffer (Akoya Biosciences). Multiplex fluorescent immunostaining was conducted following the Opal 4-color user manual (Akoya Biosciences), including a nuclear DAPI counterstain. A tracheobronchial lymph node from a cat determined to no longer have systemic infection was used simultaneously as a negative control for GFP and positive control for myeloid/histiocytic antigen and CD20. Whole slide images were acquired using a Zeiss Axio Scan Z. 1 whole slide scanner at 200× equipped with a Colibri 7 LED light source and 16 bit Orcha Flash 4.0 monochrome camera. Immunohistochemical and acquisition parameters are outlined in Table 2.

TABLE 2
Multiplex fluorescent immunohistochemistry
methodology summary

| | | | | Opal | | | |
|---|---|---|---|---|---|---|---|
| | | | | Dye | | Expo- | Opal |
| | | | | Emis- | | sure | Dye |
| Antibody | Catalog | Dilu- | AR | sion | Dichroic | Time | Dilu- |
| Target | # | tion | Order | (nm) | Filter | (ms) | tion |
| CD20 | ACR | 1:80 | 1 | 520 | 488 | 30 | 1:150 |
| | 3004 | | | | GFP | | |
| | (Biocare | | | | | | |
| | medical) | | | | | | |
| Myeloid/ | M0747 | 1:400 | 2 | 570 | 555 td | 30 | 1:150 |
| Histiocytic | (Dako) | | | | Tomato | | |
| Antigen | | | | | | | |
| GFP | A11122 | 1:200 | 3 | 690 | 647 Cy5 | 30 | 1:100 |
| | (Thermo | | | | | | |
| | Fisher | | | | | | |
| | Scientific) | | | | | | |

### Results

FeMV Uses feCD150 as a Cellular Receptor

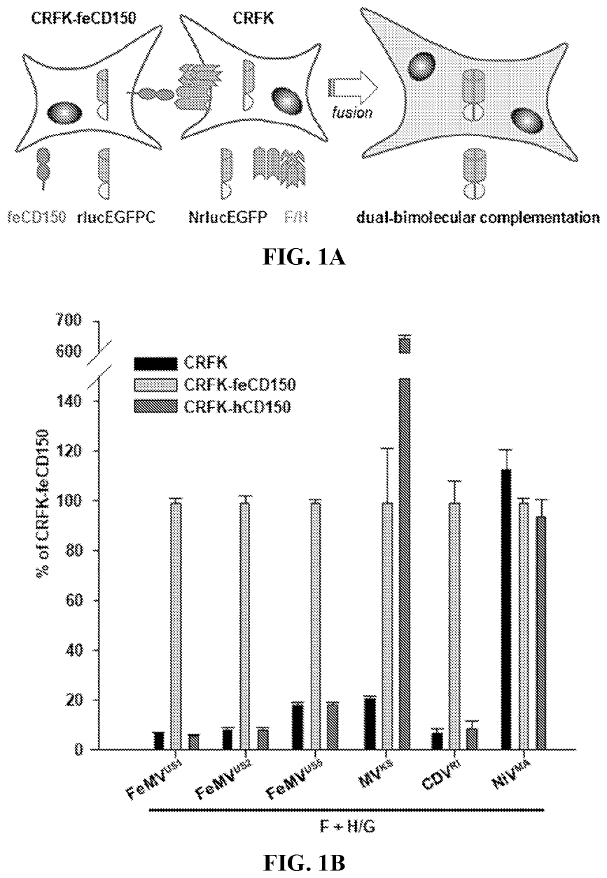

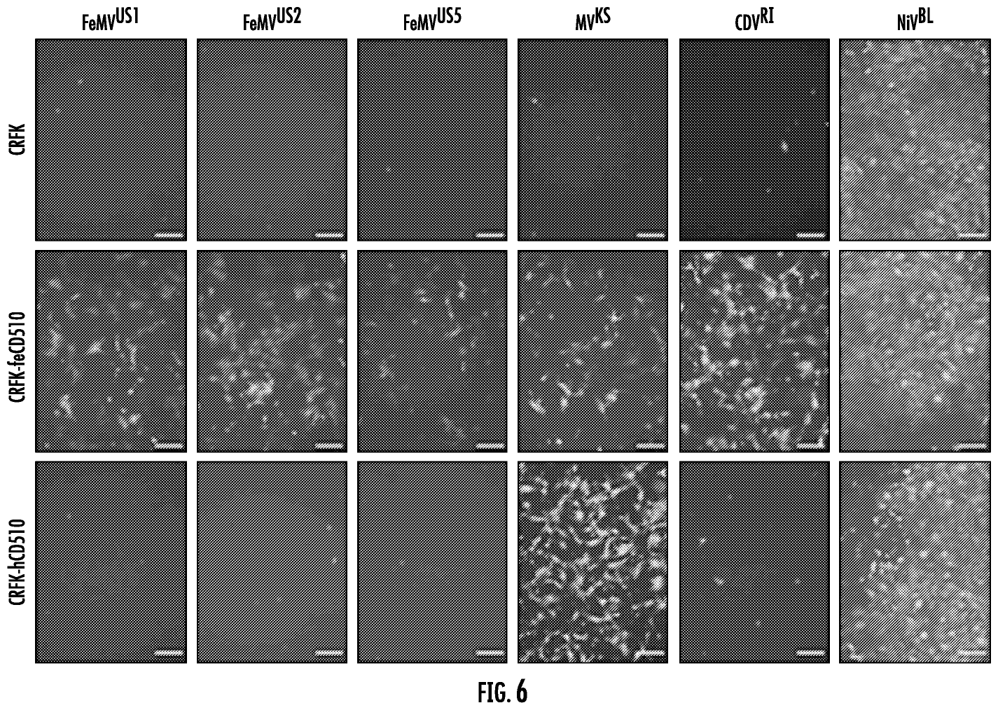

A quantitative dual-bimolecular complementation assay (FIG. 1A) was used to examine receptor usage by the F and H glycoproteins of three different FeMV strains (US1, US2 and US5). When two populations of CRFK cells were transfected no signal was generated (FIG. 6, top panels; FIG. 1B). Controls with MV and CDV glycoproteins substituted for the FeMV glycoproteins also failed to produce signal; substitution with the F and G glycoproteins from Nipah virus (NiV), which uses ephrin B2 as a receptor (Negrete, et al., Nature, 436: 401-405 (2005) and Bonaparte, et al., Proc. Nat'l Acad. Sci. USA, 102:10652-10657 (2005)), a molecule thought likely to be present on CRFK cells, acted as a positive control. In contrast when CRFK-feCD150 cells were used in the assay, signal was generated for all of the FeMV strains (FIG. 6, middle panels and FIG. 1B), and MV and CDV glycoprotein expression also led to signal generation, indicating that these morbillivirus glycoproteins can also use feCD150 as a receptor. When hCD150 was substituted for feCD150 only MV glycoprotein expression led to signal generation (FIG. 6, bottom panels; FIG. 1B), indicating that FeMV cannot use hCD150 as was already known for CDV (Bieringer, et al., PLOS One, 8, e57488 (2013).

### FeMV Glycoprotein-Induced Fusion is Dependent on Cysteine Protease Availability

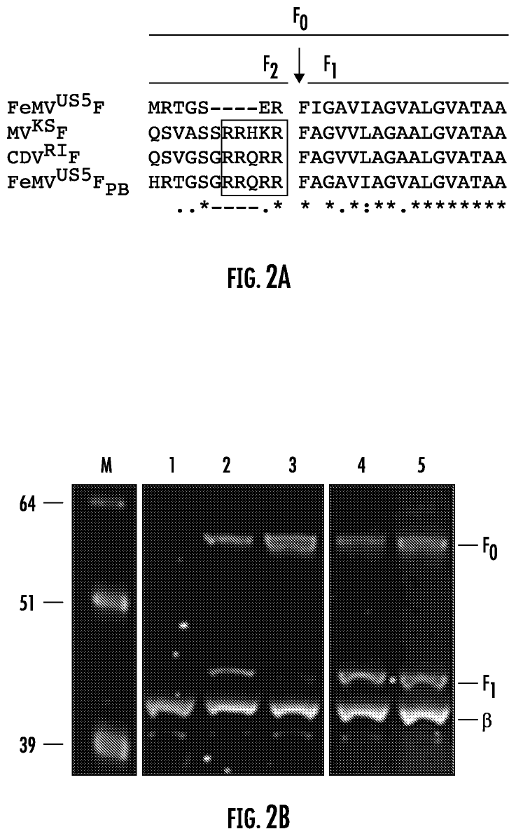

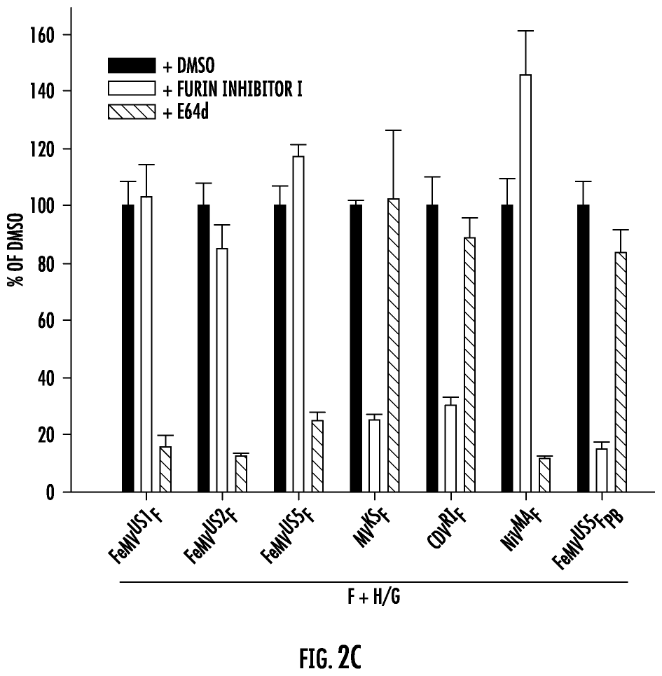

Alignment of the F glycoprotein of FeMV^{US5} with those of MV^{KS} and CDV^{RI} reveals only a single basic residue (Arg/R) at the putative cleavage site (FIG. 2A). The closely related paramyxoviruses NiV and Hendra virus (HeV) [see (Nambulli, et al., Opin. Virol., 16 (2016)) and (Woo, et al., Proc. Nat'l Acad. Sci. USA, 109:5435-5440 (2012)) for phylogenetic trees] also contain monobasic cleavage signals in their F glycoproteins and have been shown to use the cysteine proteases, cathepsins, for their cleavage (Pager, et al., Virology, 346:251-257 (2006); Pager, et al., J. Virol., 79:12714-12720 (2005); and Diederich, et al., J. Virol., 86:3736-3745 (2012)). Lysates from CRFK cells transfected with a vector expressing AU1-tagged FeMV^{US5}F in the absence or presence of the pan-cysteine protease inhibitor E64d were prepared and analyzed (FIG. 2B). Unprocessed F₀ and the F₁ subunit could be detected in cells treated with DMSO as a control (lane 2) indicating that FeMV^{US5}F was processed as predicted from the sequence alignment (FIG. 2A). F₀ was detected in cells treated with E64d (lane 3), but the post-processing F₁ subunit was not indicating E64d prevented FeMV^{US5}F processing, and showing that a cysteine protease was responsible.

FeMV^{US5}F_{PB} was generated by inserting a polybasic cleavage signal into the FeMV^{US5}F glycoprotein (FIG. 2A) based on the CDV^{RI}F sequence (accession number AMH87497.1) since this had the most sequence similarity to FeMV^{US5}F in that region. Lysates from CRFK cells transfected with a vector expressing AU1-tagged FeMV^{US5}F_{PB} in the absence or presence of E64d were prepared and analyzed (FIG. 2B). In cells treated with DMSO (lane 4) F₀ and the F₁ subunit could be detected. In cells treated with E64d (lane 5) both F₀ and F₁ subunit could still be detected, indicating that E64d no longer prevented F₀ glycoprotein processing and that a cysteine protease was no longer responsible for FeMV^{US5}F_{PB} processing.

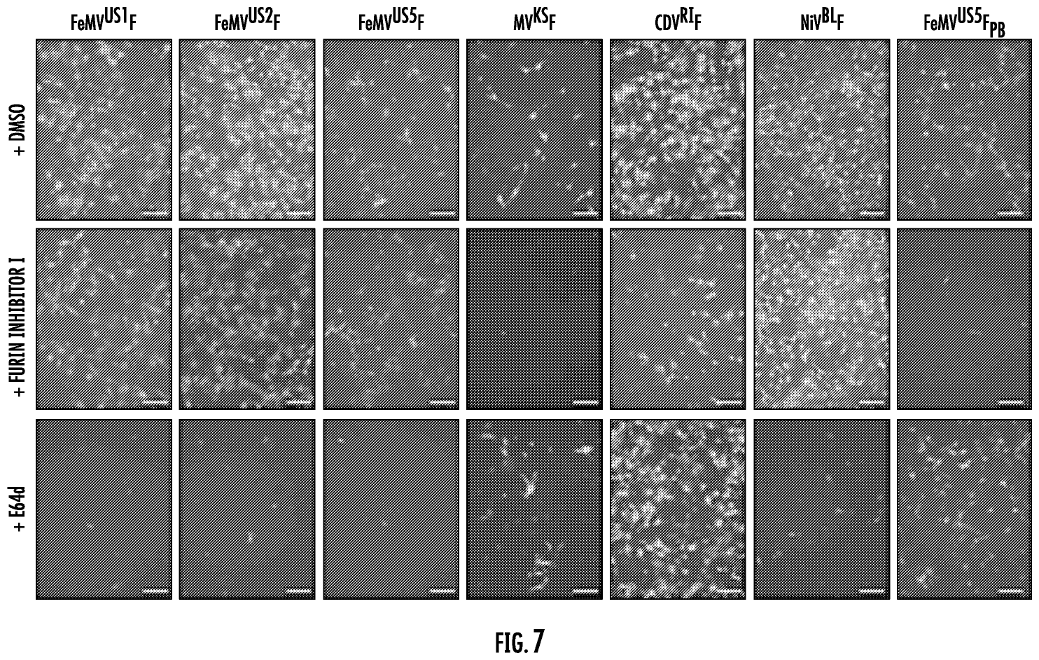

This was investigated this further by performing the bimolecular complementation assay in the presence of E64d cysteine protease inhibitor or furin inhibitor I. In the presence of DMSO (FIG. 7, top panels and FIG. 2C) all viral glycoproteins induced expected levels of fusion. In the presence of furin inhibitor I (FIG. 7, middle panels and FIG. 2C), FeMV glycoprotein-induced fusion was unaffected while the control MV^{KS} and CDV^{RI} glycoprotein-induced fusion (furin dependent) was reduced. In the presence of E64d (FIG. 7, bottom panels and FIG. 2C), FeMV glycoprotein-induced fusion was significantly reduced, as was control NiV^{MA} glycoprotein-induced fusion (cathepsin dependent). In assays using the FeMV^{US5}F_{PB} protein, E64d treatment (FIG. 7, bottom panel and FIG. 2C) no longer affected the fusion activity, while furin inhibitor I treatment (FIG. 7, middle panel and FIG. 2C) significantly reduced fusion, indicating that switching the protease dependence of the protein had been successful.

### Generation of an FeMV Reverse Genetics System

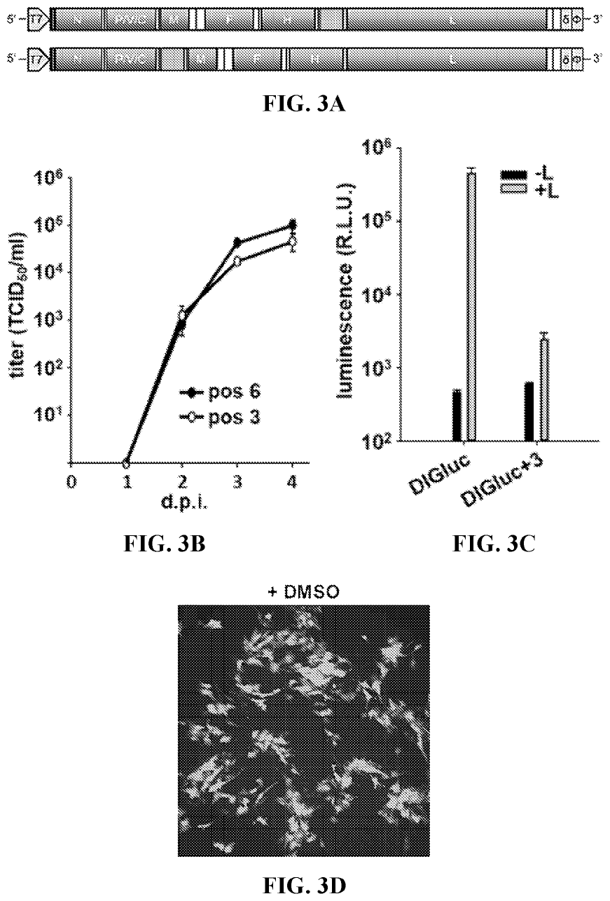

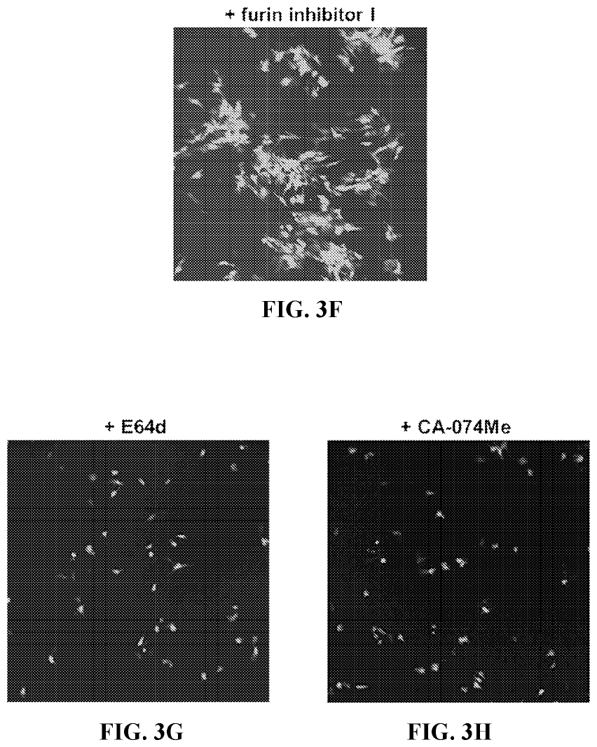

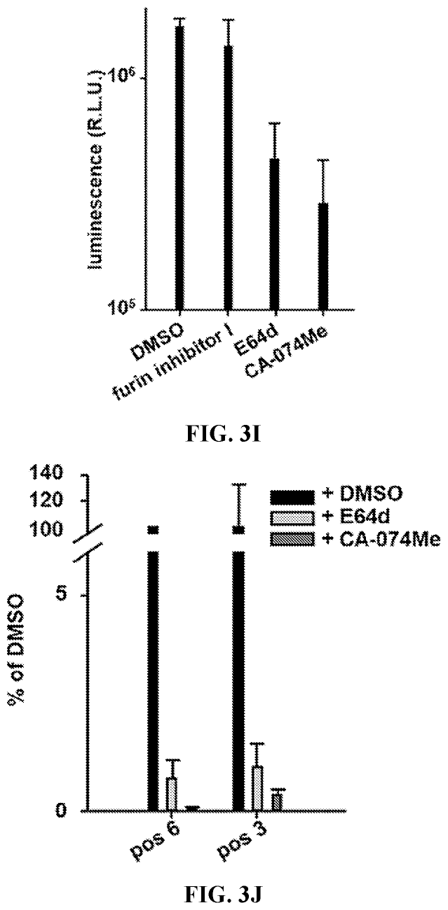

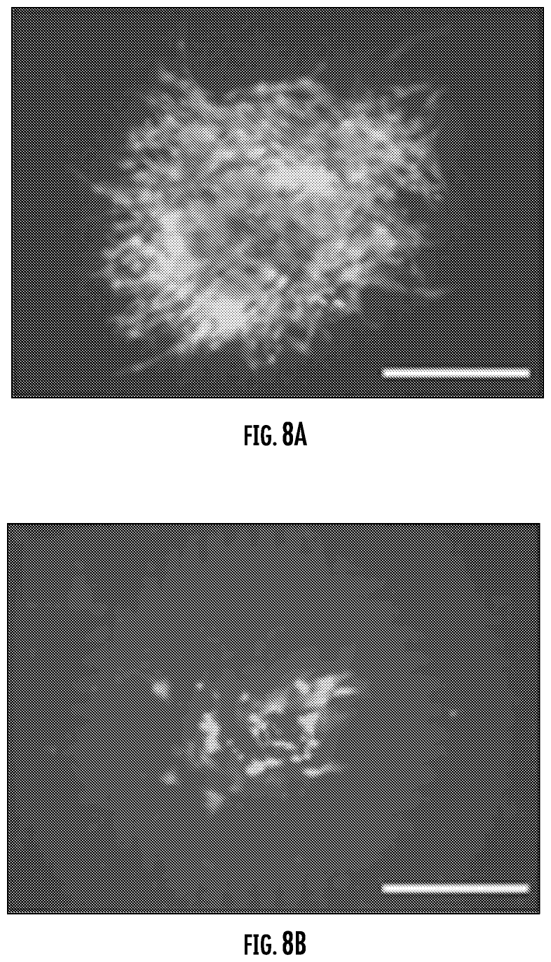

The full genomic sequence of FeMV^{US5} using was determined using primers (Table 1) to generate cDNA from clinical material and generate PCR amplicons which were purified and consensus sequenced. Importantly this entire sequence was derived directly from a clinical sample and not from a virus that had been isolated and grown in cell culture. Large amplicons from the FeMV^{US5} sequence determination were subcloned, a cloning strategy devised, and a full-length genomic pFeMV^{US5} clone was assembled sequentially. This molecular clone was modified to include an additional transcription unit (ATU) encoding Venus fluorescent protein between the H and L genes to generate pFeMV^{US5}Venus (6) or between the P and M genes to generate pFeMV^{US5}Venus (3) (FIG. 3A). Viruses were recovered using CRFK-feCD150 cells to ensure the virus did not evolve to use an unnatural entry pathway or accumulate spurious mutations. The Venus protein allowed infected cells to be identified by fluorescence microscopy even when cytopathic effect is not readily identifiable by phase microscopy (FIGS. 8A-8B). This was vitally important given the cell-associated growth properties of FeMV. Virus stocks were generated and the growth kinetics in CRFK-feCD150 cells were determined (FIG. 3B). Both viruses grew similarly for the first four days post-infection.

### FeMV Obeys the ‘Rule of Six’

The FeMV^{US5} sequence and the resultant recombinant viruses that were generated follow the ‘rule of six’ (Kolakofsky, et al., J. Virol., 72:891-899 (1998)). To investigate whether this is a requirement for FeMV, a minigenome, rFeMV^{US5}DIGluc, was generated which had the coding sequence for Gaussia luciferase (Gluc), as reporter gene, surrounded by the FeMV^{US5} 3′ and 5′ non-coding termini. rFeMV^{US5}DIGluc adhered to the ‘rule of six’. When tested in minigenome assays this minigenome expressed a high level of Gluc activity (FIG. 3C, +L). When the L protein expressing vector was omitted from the assay 3 logs less Gluc activity was detected (FIG. 3C,-L) indicating that minigenome replication and transcription were necessary to produce Gluc signal. rFeMV^{US5}DIGluc was modified by removing a second stop codon at the end of the Gluc open reading frame to generate rFeMV^{US5}DIGluc+3. rFeMV^{US5}DIGluc+3 does not adhere to the ‘rule of six.’ When this minigenome was tested Gluc activity was dramatically reduced (FIG. 3C, +L versus-L) indicating that ‘rule of six’ adherence is a requirement for efficient FeMV^{US5} replication.

### FeMV is a Cathepsin-Dependent Morbillivirus

Next, whether rFeMV^{US5}-induced cell-to-cell fusion was, like H and F glycoprotein-induced fusion, dependent on a cysteine protease was investigated. CRFK-feCD150 cells were infected with rFeMV^{US5}Venus (6) in the presence of DMSO as a control, furin inhibitor I, cysteine protease inhibitor E64d, or cathepsin B/L inhibitor CA-074Me. After five days, the rFeMV^{US5}Venus (6) infection in the presence of DMSO or furin inhibitor I had spread significantly (FIGS. 3D-3F). However, the infection was limited to single cells with no spread from the initially infected cell in the presence of cysteine protease (FIG. 3G) or cathepsin (FIG. 3H) inhibitors. To quantify these differences CRFK-feCD150 cells were transfected with pCG-NrlucEGFP, and 24 hours later were infected with rFeMV^{US5}Venus (6) and inhibitors were added. After another 24 hours these cells were overlaid (in the presence of inhibitor) with a population of CRFK-feCD150 cells that had been transfected with pCG-rlucEGFPC 48 hours earlier. After incubation with inhibitors for 2 days, monolayers were assessed for luciferase activity (FIG. 3I). As expected, there was no difference in luciferase activity in cells treated with furin inhibitor versus the DMSO control, whereas activity was reduced in cells treated with cysteine protease or cathepsin inhibitors.

Triplicate CRFK-feCD150 monolayers were infected with rFeMV^{US5}Venus (6) or rFeMV^{US5}Venus (3) in the presence of DMSO, E64d cysteine protease inhibitor or CA-074Me cathepsin inhibitor. After four days released virus was quantified (FIG. 3J). The presence of cysteine protease or cathepsin inhibitors reduced the infectious virus titers by two logs, indicating that the inhibitors prevented efficient assembly and egress of infectious virions.

### FeMV Causes a Morbillivirus-Like Disease in the Natural Host

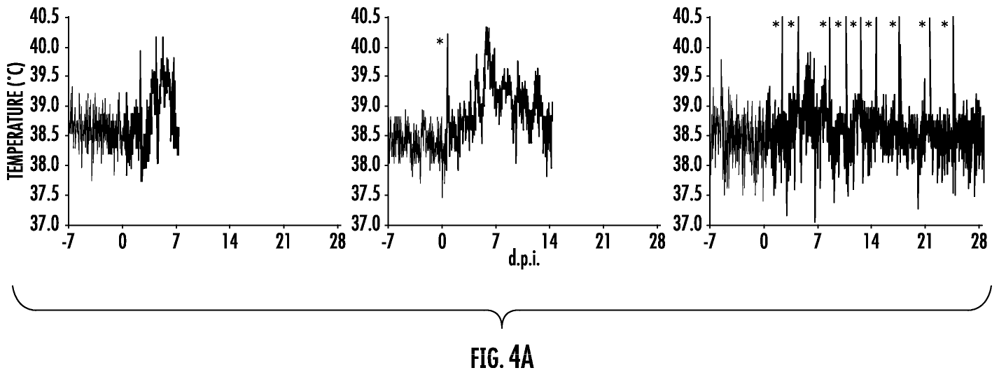

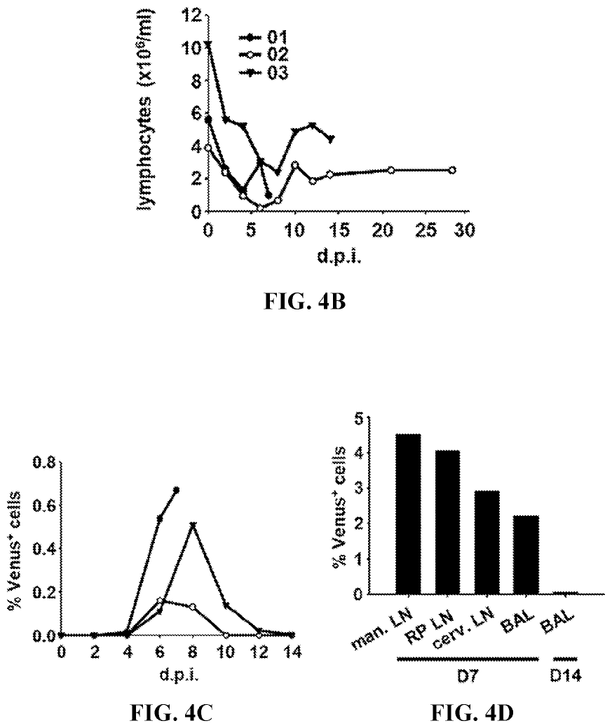

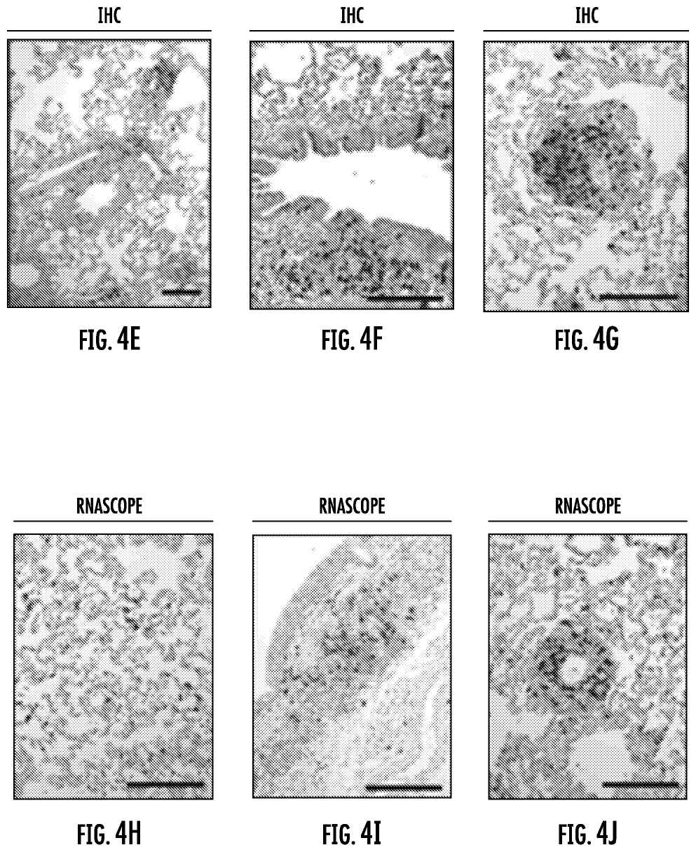

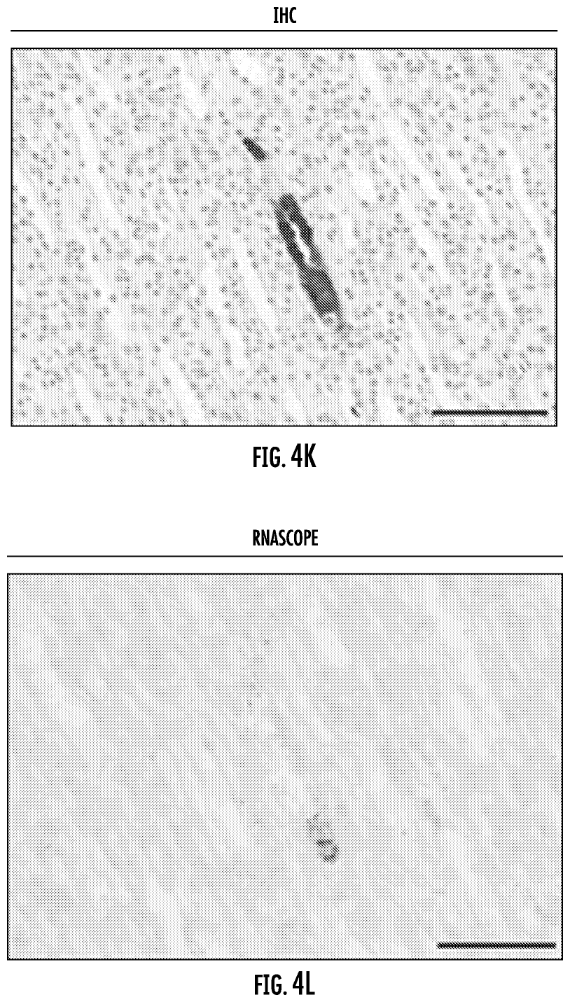

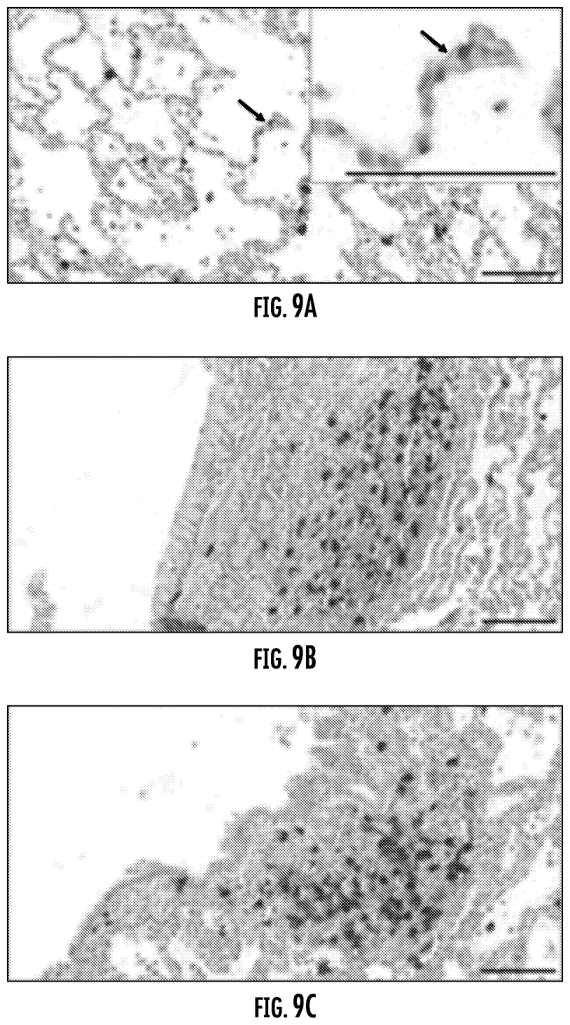

Nothing is known about the primary route of infection or disease progression of FeMV-infected animals. Cats were infected with rFeMV^{US5}Venus (6) and rFeMV^{US5}Venus (3). Animals were pre-implanted (intraperitoneally) with a temperature data logger and blood samples were collected every 2 d.p.i. until 14 d.p.i. At 7 d.p.i., 14 d.p.i. and 28 d.p.i., one cat was euthanized and a full necropsy was performed to determine the acute phase pathogenesis of the virus. Data loggers recorded an increase in body temperature after infection, peaking at 5 d.p.i. (FIG. 4A). All animals developed lymphopenia early after infection (FIG. 4B). White blood cells were isolated from the blood samples and the percentage of virus⁺ (Venus⁺) cells were determined by flow cytometry (FIG. 4C). All animals had detectable Venus⁺ white blood cells at 6-10 d.p.i., with concomitant virus isolation from these samples (Table 1), after which the infection cleared from the blood. At necropsy, Venus⁺ cells could be detected in single cell suspensions from lymph nodes, and in the bronchoalveolar lavage (BAL) sample at 7 d.p.i. (FIG. 4C). By 14 d.p.i. Venus⁺ cells were barely detectable in the BAL, and not detectable in the lymph nodes. Histopathological assessment of tissues collected at each necropsy confirmed disease and identified the peak of virus detection at 7 d.p.i. In the lungs, Venus protein and viral RNA could be detected in the interstitium (FIGS. 4E and H, and FIG. 9A), bronchial tissue (FIGS. 4F and I, and FIG. 9B) and in bronchus associated lymphoid tissue (FIGS. 4G and J, and FIG. 9C) at 7 d.p.i, and virus was isolated from lung tissue and BAL at this time point (Table 3).

TABLE 3
Summary of days post-infection when virus
was isolated from clinical samples

| | | Throat | | Broncho- | |
|---|---|---|---|---|---|
| | White blood | and nose | | alveolar | |
| Animal | cells ² | swabs ³ | Urine ⁴ | lavage ⁵ | Lung ⁵ |
| 01 ¹ | 4, 6, 7 | — ⁶ | — ⁶ | 7 | 7 |
| 02 ¹ | 8 | — ⁶ | 12, 21, 28 | — ⁶ | — ⁶ |
| 03 ¹ | 4, 6, 8, 10, 12, 14 | — ⁶ | 14⁷ | — ⁶ | — ⁶ |

¹ Animals 01, 02 and 03 were euthanized at 7, 28, and 14 days post-infection respectively
² Samples were collected from living animals at 2, 4, 6, 8, 10, 12, 14, and 21 days post-infection and from each animal at necropsy
³ Samples were collected from living animals at 2, 6, 12, 17, and 21 days post-infection and from animal 02 at necropsy
⁴ Samples were collected from living animals at 6, 12, and 21 days post-infection and from each animal at necropsy
⁵ Samples were collected at necropsy only
⁶ Virus was not isolated from any samples collected
⁷No sample obtained at 12 days post-infection

### FeMV Targets the Kidneys Later in Infection

In addition to the samples outlined above urine samples were also collected from all living animals at 6, 12, and 21 d.p.i. and from all animals at necropsy. Virus was isolated from all animal 02 samples from 12 d.p.i. onwards and from animal 03 at necropsy (14 d.p.i.; Table 1). No virus was isolated from any animal urine at the earlier time points, when virus detection peaked in the white blood cells (FIG. 4C) and in BAL (FIG. 4D). Histopathological assessment of hematoxylin and eosin-stained kidney sections indicated the presence of lymphoplasmacytic lesions and pelvitis in the 14 and 28 d.p.i. sections but not in the 7 d.p.i. sections. Based on these observations, kidney sections were assessed for presence of virus, with Venus protein (indicating infected cells; FIG. 4K) and viral RNA (FIG. 4L) detected in medullary tubule epithelium in the 28 d.p.i. sections. Virus was not detected in the 7 or 14 d.p.i. sections.

Lymphoid Tissues are Targeted During Acute rFeMV Infection

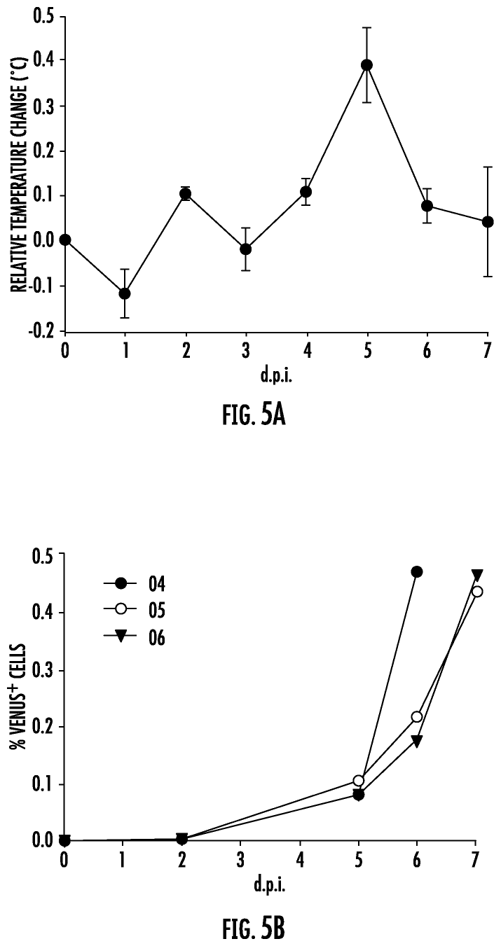

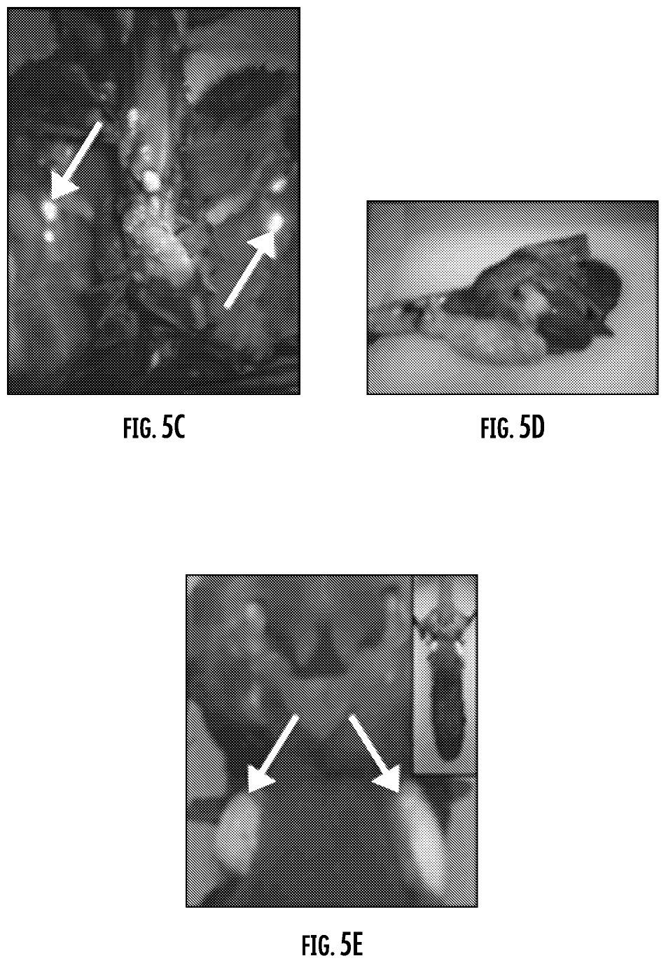

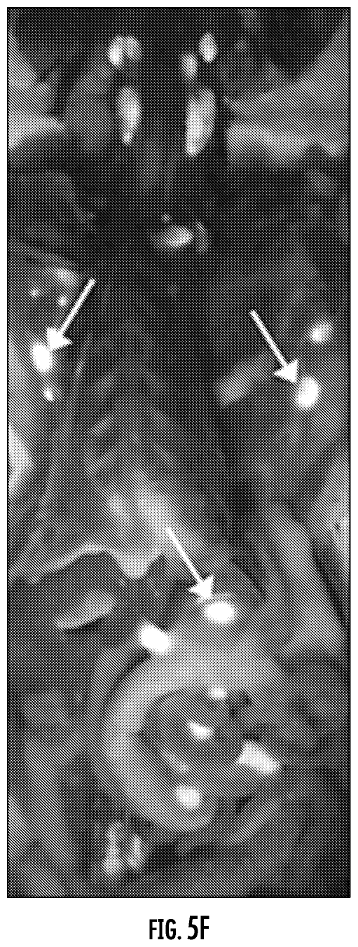

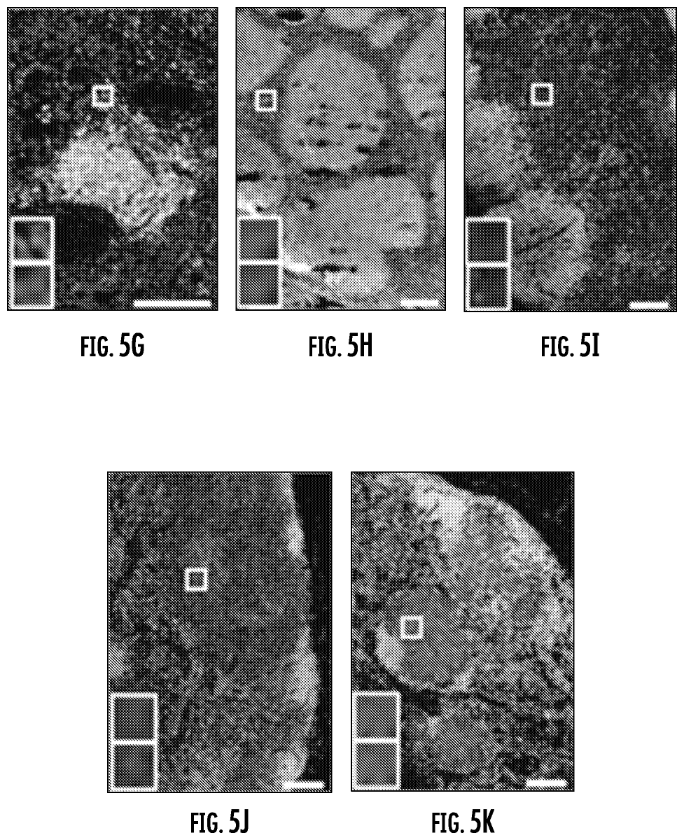

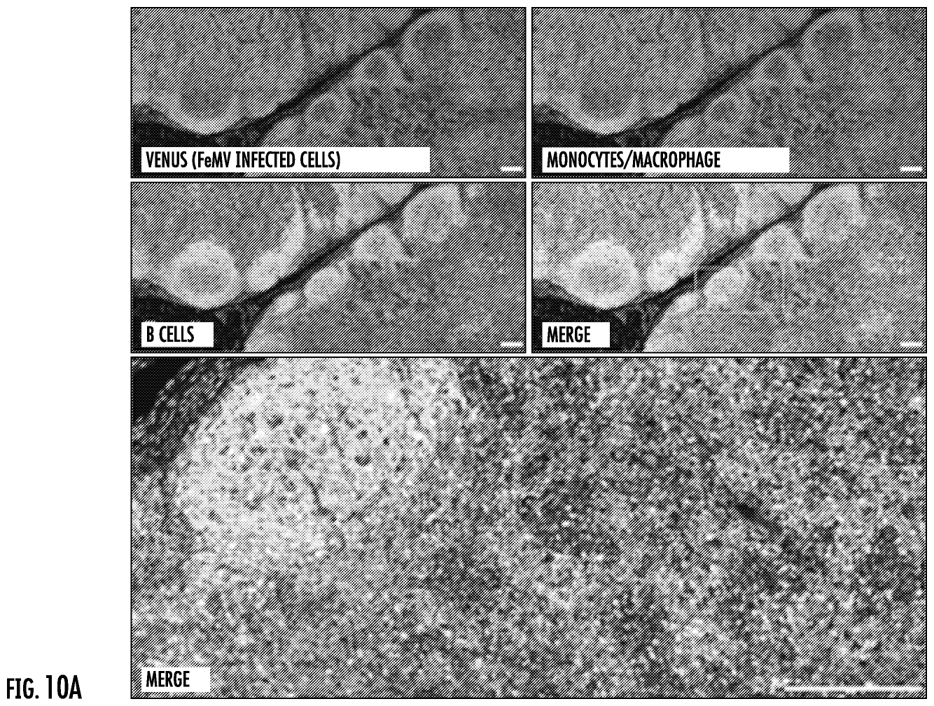

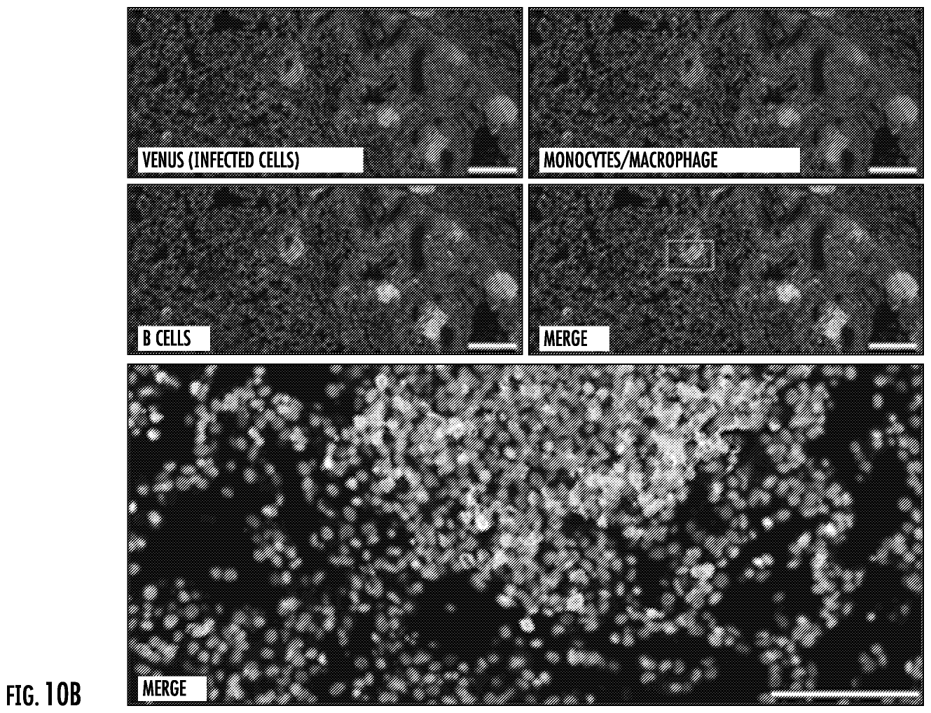

Three additional cats were infected with rFeMV^{US5}Venus (6) and rFeMV^{US5}Venus (3) to examine the peak of infection. Animals were pre-implanted (subcutaneous) with a data logger and blood samples were collected from all animals at 2, 5, and 6 d.p.i. and two animals at 7 d.p.i. All animals were euthanized at 7 d.p.i. and full necropsies were performed to characterize the acute phase pathogenesis of the virus. Infections proceeded similarly, temperature increases peaked at 5 d.p.i. (FIG. 5A) and similar percentages of Venus⁺ cells detected in the blood at equivalent time points (FIG. 5B). Macroscopic imaging confirmed lymphotropism of the acute infection, all lymph nodes were highly infected (FIGS. 5C and 5F), thymus (FIGS. 5C and 5D) and tonsils (FIG. 5E) fluorescing brightly following macroscopic bioimaging. Multiplex fluorescence immunohistochemistry in formalin fixed paraffin embedded lung and lymph node tissues was performed to identify the infected cell populations. Antibodies which are verified for use in cat tissue are limited but B-cell (CD20) and monocyte/macrophage (myeloid/histiocyte antigen) markers which were functional were identified. An antibody against Venus was also included to mark infected cells. In all tissues examined the majority of Venus⁺ cells were monocytes/macrophages (FIG. 5G-5K, FIG. 10A, and FIG. 10B), however not all monocytes/macrophages were Venus⁺. Venus⁺ B-cells were not identified in any of the tissues examined.

### Discussion

FeMV Uses feCD150 as an Entry Receptor

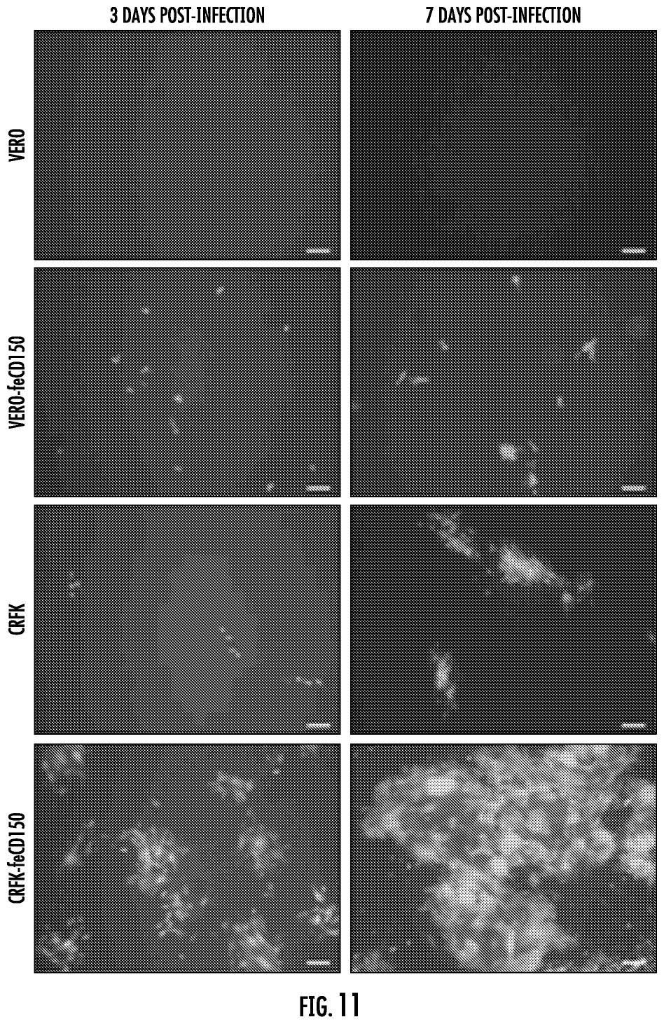

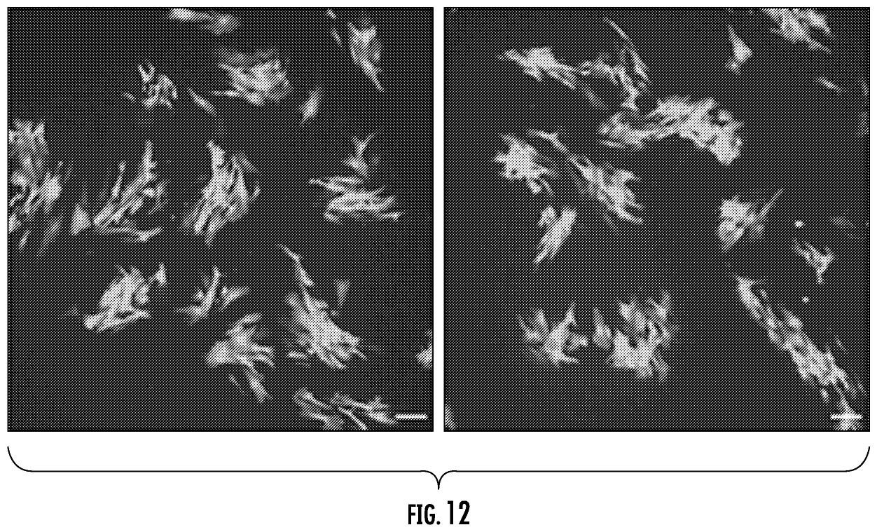

Based on a lack of conservation of residues known to be important for morbillivirus H interaction with CD150 (28-34) is was hypothesized that FeMV could not use CD150 as a receptor. However, when FeMV F and H glycoproteins were expressed in feCD150-positive cells they caused cell-to-cell fusion (FIG. 1B and FIG. 6). MV and CDV also bind feCD150 and induce cell-to-cell fusion. Similarly, rFeMV^{US5}Venus (6) and rFeMV^{US5}Venus (3) also required feCD150 expression to infect and spread in CRFK cells. An attempt was made to analyze the growth kinetics of these viruses in CRFK cells lacking feCD150 even though they occasionally entered cells at very low efficiency cell-to-cell spread was limited and infectious virus was never recovered (FIG. 11). The ability of wild-type FeMV to enter CRFK cells at low efficiency likely explains how some groups have been able to culture FeMV after prolonged blind passage, presumably concomitant with the accumulation of adaptive mutations, in cells lacking a CD150 receptor (Woo, et al., Proc. Nat'l Acad. Sci. USA, 109:5435-5440 (2012); Sakaguchi, et al., J. Gen. Virol., 95:1464-1468 (2014); Sieg, et al., Viruses, 11 (2019); Sieg, et al., Genome Announc., 6 (2018)) which occurs during wild-type MV passage in Vero and chicken fibroblast cells (Xin, et al., Intervirology, 54:217-228 (2011); Kouomou, et al., The Journal of Virology, 76:1505-1509 (2002); Rima, et al., J. Gen. Virol., 78:97-106 (1997)) and led to adaption of that virus to use CD46 as a receptor (Naniche, et al., The Journal of Virology, 67:6025-6032 (1993); Dörig, et al., Cell, 75:295-305 (1993)). Crucially, the virus that formed the base of the reverse genetics system described herein was sequenced directly from a clinical sample (urine) and had never been isolated or passaged in laboratory cells; it is critical that such a virus, which has had no opportunity to adapt to alternative receptor usage, is used for the type of pathogenesis studies described herein.

### FeMV is a Unique Morbillivirus Employing a Cathepsin Protease for F Glycoprotein Processing

All paramyxovirus F glycoproteins are expressed first as an inactive precursor (F₀) which is processed proteolytically by the ubiquitous cellular protease furin (Watanabe, et al., The Journal of Virology, 69:3206-3210 (1995)) to produce disulfide-linked active F₁ and F₂ subunits. Processing exposes the hydrophobic fusion peptide and biologically active F₁ and F₂ subunits in complex with the H glycoprotein are transported to lipid rafts on the plasma membrane where virions are assembled (Aguilar, et al., Curr. Clin. Microbiol. Rep., 3:142-154 (2016)).

Until the discovery of FeMV, all morbillivirus F glycoprotein sequences contained a polybasic cleavage signal at the predicted furin cleavage site. Alignment of the FeMV F glycoprotein sequences of FeMV, MV and CDV identified the highly conserved hydrophobic fusion peptide at the end of F₁. However, surprisingly an upstream polybasic signal was absent and only a single basic residue was present at the predicted cleavage site.

It has been reported that FeMV uses a cellular trypsin-like protease to cleave F₀ at the monobasic cleavage signal (Woo, et al., Proc. Nat'l Acad. Sci. USA, 109:5435-5440 (2012)).

When the fusion assays were first performed with the FeMV glycoproteins transfected into CRFK-feCD150 cells, it was observed that the induction of cell-to-cell fusion did not require the addition of exogenous trypsin, and addition of such protease did not enhance fusion; similarly it was subsequently reported that addition of trypsin during virus titration in CRFK cells did not augment the resultant virus titers (Koide, et al., J. Vet. Med. Sci., 77:565-569 (2015)). This suggested that F₀ was being cleaved efficiently using an endogenous protease expressed in the cells.

A quantitative dual-bimolecular complementation assay was used in the presence of protease specific inhibitors to show that furin was not the protease responsible, and that a cysteine protease was. The cysteine protease inhibitor E64d and cathepsin B/L inhibitor CA-074Me (Montaser, et al., Biol. Chem., 383:1305-1308 (2002) also prevented cell-to-cell fusion and spread by the recombinant viruses rFeMV^{US5}Venus (6) and rFeMV^{US5}Venus (3) in CRFK-feCD150 cells. Since replication of the viruses must occur in infected cells before green fluorescence can be detected, it is evident by comparing the foci of infection in FIG. 3E with the number of infected cells in FIGS. 3G and 3H that in the presence of E64d or CA-074Me viruses can enter and initially replicate in cells equivalently to the no inhibitor control. The block in cell-to-cell fusion must occur due to the inability of de novo synthesized fusion protein to be cleaved and trafficked to the plasma membrane. These data also indicate that the F₀ glycoprotein is not cleaved during entry of the virus to the cell as is the case with, for example, Ebola virus (Chandran, et al., Science, 308:1643-1645 (2005)) or some coronaviruses (Millet, et al., Virus Res., 202:120-134 (2015)).

### FeMV Causes an Acute Morbillivirus-Like Disease in the Natural Host

Cats (Felis catus) are naturally infected by FeMV and are therefore the ideal species to examine FeMV infection, pathogenesis and transmission. Initially three cats were infected with FeMV expressing fluorescent protein to examine the time course of infection. The ability of a virus to produce green fluorescence in infected cells is a powerful means to track virus spread in animals, and identify very small numbers of infected cells. Animals were euthanized at 7, 14, and 28 d.p.i. to examine spread of the virus over time and the tissues which are targeted. The sampling and necropsy time points were chosen based on the disease courses of CDV in ferrets (Vries, et al., PLOS Pathog., 13: e1006371 (2017)) and MV in macaques (El Mubarak, et al., J. Gen. Virol., 88:2028-2034 (2007)).

All animals showed an increase in temperature, peaking at 5 d.p.i., which is reminiscent of CDV-induced temperature increases in ferrets (Vries, et al., PLOS Pathog., 13: e1006371 (2017)). Virus was detected in the white blood cells (WBCs), and animals developed lymphopenia, both hallmarks of morbillivirus disease in susceptible hosts. In both the animals that remained alive after 7 d.p.i. the lymphocytes numbers recovered, although they did not reach pre-infection levels during the time course of the experiment, a pattern also observed in MV and CDV infections. Lymphodepletion contributes to the long term immunosuppression seen after morbillivirus infection. At peak, the percentage of Venus⁺ WBCs were very low compared to those seen in MV-infected macaques, and particularly in CDV-infected ferrets where cell populations are decimated, leading to a propensity for secondary infections and frequent necessity to euthanize animals by 14-16 d.p.i. One possible explanation for this is the availability of cathepsin B in the peripheral blood cells; in humans, levels are extremely low in CD19⁺ B cells, and CD4⁺ and CD8⁺ T cells which are all major targets for MV and CDV in animal models. Cathepsin B levels are significantly higher in CD14⁺ monocytes, which are present at much lower levels in the cat blood (2.4-7.1% at day 0) compared to the lymphocytes (40.9-45.8% at day 0). Monocytes are also significant target cells for MV in humans that is not recapitulated in macaque infections. The low levels of infected cells in WBCs, even at the peak of acute infection, may also explain why other groups have been unsuccessful in detecting virus in blood samples.

Surprisingly, the virus was not isolated from nose or throat swabs at any time point assayed; virus shedding from the respiratory tract peaks at 7-11 d.p.i. in MV-infected macaques and increases during the second week of infection in CDV-infected ferrets. However, the virus from was isolated from urine at later time points.

At necropsy virus was detected in cells purified from lymph nodes and a BAL sample at 7 d.p.i. Viral antigen and RNA were also both abundant in the lungs at this time point. Bronchi and bronchiole epithelium never displayed immunoreactivity/probe hybridization, and only rare alveolar type 1 pneumocytes were impacted with the overwhelming majority of signal observed in perivascular, peribronchiolar, interstitial, and alveolar mononuclear infiltrates. Phenotyping the infected cells in the lymph nodes and lung indicated the majority of the Venus⁺ infected cells co-stained for monocyte/macrophage marker, with minimal to absent co-staining for B-cell marker. Unfortunately, a specific T-cell marker could not be identified that worked efficiently in feline tissues. However limited preliminary analysis and the localization of infected cells suggest that T-cells are not a major target for FeMV in lymphoid/lung tissues. These findings are at variance to what is seen in lymphoid tissue infection with MV in macaques and CDV in ferrets where B- and T-cells are abundantly infected. However, macrophages/dendritic cells were identified as a major target for MV in lung. Interestingly it was previously shown that a feline macrophage cell line, Fcwf-4, was highly susceptible to FeMV, and this was corroborated by infection of these cells with rFeMV^{US5}Venus (3) (FIG. 12). IHC performed on lymph node sections from naturally infected FeMV⁺ cats also identified infected macrophages. It has previously been shown that feline primary pulmonary epithelial cells are susceptible to a closely related FeMV in vitro. The in vivo study described herein extends this observation showing that although some FeMV-infected pneumocytes could be detected in lung (FIG. 9A), the extensive alveolar epithelium disruption reported for MV was not observed, which could explain why virus was not isolated from the nose and throat swabs.

FeMV was originally detected in, and isolated from, cat urine samples. The virus was isolated from cats infected with rFeMV^{US5}. rFeMV^{US5} was shed in the urine from 12 d.p.i. and was still present at necropsy in the urine of the one cat that was allowed to progress to 28 d.p.i. Virus could not be isolated from any cat urine sample collected at 6 d.p.i. MV can also be isolated from the urine of measles patients after the appearance of rash and CDV can be detected in the urine of naturally infected dogs where it is present at high viral load. At necropsy rFeMV^{US5} was detected in the renal medullary tubule epithelium by IHC and corroborated this by detecting RNA in a serial section. This detection is in good agreement with analysis of kidney sections from naturally infected cats where FeMV antigen was detected in renal tubular cells. It has also been previously shown that feline primary kidney cells are susceptible to FeMV and that epithelial cells are the primary target (Sieg, et al., Viruses, 11 (2019)). The facts that infectious FeMV can be readily isolated from cat urine for a prolonged period [16 days in this study to months (Sieg, et al., Viruses, 11 (2019))], and that virus from nose or throat swabs was not isolated at any time in the study raise the intriguing question of how FeMV is transmitted, and whether this differs from the respiratory transmission used by other morbilliviruses.

In these studies, it has been shown that FeMV uses feCD150 as a cellular receptor and employs a unique protease for F glycoprotein processing. The fluorescent protein expressing rFeMV of an aspect of the invention has been used to illuminate viral pathogenesis in the cat following infection via a natural route.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments and aspects of this invention are described herein. Variations of those preferred embodiments and aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of producing recombinant feline morbillivirus (FeMV), the method comprising using reverse genetics.

2. The method of claim 1, the method comprising:
a. extracting FeMV RNA from an isolated FeMV positive sample;
b. sequencing the extracted FeMV RNA;
c. aligning the extracted FeMV RNA sequences to each other;
d. preparing consensus sequences from the aligned FeMV RNA sequences;
e. assembling the consensus sequences; and
f. preparing full-length recombinant FeMV based on the assembled consensus sequences using amplicons, synthetic DNA, or a combination thereof.

3. A method of producing recombinant feline morbillivirus (FeMV), the method comprising:
a. extracting FeMV RNA from an isolated FeMV positive sample;
b. generating cDNAs from the FeMV RNA using primers that specifically hybridize to the FeMV RNA;
c. generating cDNA PCR amplicons from the cDNAs using primers that specifically hybridize to the cDNAs to produce cDNA PCR amplicons;
d. amplifying genomic and antigenomic termini of the FeMV RNA by rapid amplification of cDNA ends (RACE) using one or more RACE primers to produce RACE PCR amplicons;
e. purifying the cDNA PCR amplicons of step (c) and the RACE PCR amplicons of step (d) to produce purified DNAs;
f. sequencing the purified DNAs to produce consensus sequences;
g. assembling the consensus sequences to produce a full-length FeMV genome; and
h. assembling the full-length FeMV genome in a plasmid.

4. The method of claim 3, further comprising:
i. transfecting cells to express feline CD150 (feCD150) and at least one feline cysteine protease to produce precursor producer cells;
j. introducing T7 RNA polymerase into the precursor producer cells by transfection or infection; and
k. transfecting the precursor producer cells with FeMV nucleo-(N), phospho-(P) and large (L) proteins and the plasmid of (h) to produce producer cells.

5. The method of claim 4, wherein the precursor producer cells are Crandell Rees feline kidney cells expressing feline CD150 (CRFK-feCD150).

6. The method of claim 4, wherein the producer cells are infected using Modified Vaccinia virus Ankara (MVA).

7. The method of claim 4, further comprising:
l. propagating the producer cells to produce the recombinant FeMV.

8. The method of claim 3, wherein the primers that specifically hybridize to the FeMV RNA comprise at least one forward primer with at least 90% identity to SEQ ID NOs: 1, 3, 7, 9, 11, 13, 15, 17, 19, 20, 22, 24, 25, 27, 30, and 33.

9. The method of claim 3, wherein the primers that specifically hybridize to the FeMV RNA comprise at least one forward primer comprising SEQ ID NOs: 1, 3, 7, 9, 11, 13, 15, 17, 19, 20, 22, 24, 25, 27, 30, and 33.

10. The method of claim 3, wherein the primers that specifically hybridize to the FeMV RNA comprise at least one reverse primer with at least 90% identity to SEQ ID NOs: 1, 2, 4, 5, 8, 12, 14, 16, 18, 6, 23, 26, 28, and 31.

11. The method of claim 3, wherein the primers that specifically hybridize to the FeMV RNA comprise at least one reverse primer comprising SEQ ID NOs: 1, 2, 4, 5, 8, 12, 14, 16, 18, 6, 23, 26, 28, and 31.

12. The method of claim 3, wherein the primers that specifically hybridize to the FeMV RNA comprise at least one cDNA primer with at least 90% identity to SEQ ID NOs: 1, 3, 6, 10, 12, 21, 29, and 32.

13. The method of claim 3, wherein the primers that specifically hybridize to the FeMV RNA comprise at least one cDNA primer comprising SEQ ID NOs: 1, 3, 6, 10, 12, 21, 29, and 32.

14. The method of claim 3, wherein at least one consensus sequence has at least 90% identity to SEQ ID NOs: 34 and 35.

15. The method of claim 3, wherein at least one consensus sequence comprises SEQ ID NOs: 34 and 35.

16. The method of claim 1, wherein the recombinant FeMV comprises genes N, P/V/C, M, F, H, and L.

17. The method of claim 1, wherein the recombinant FeMV comprises genes N, P/V/C, M, F, H, and L in arrangement 3′-N-P/V/C/-M-F-H-L-5′.

18. The method of claim 1, wherein the recombinant FeMV further comprises an additional transcription unit (ATU) encoding a detectable reporter protein.

19. The method of claim 18, wherein the ATU is located between the H and L genes of the recombinant FeMV.

20. The method of claim 18, wherein the ATU is located between the P and M genes of the recombinant FeMV.

21. The method of claim 18, wherein the ATU is located 3′ to the N gene of the recombinant FeMV.

22. The method of claim 2, wherein the FeMV sample is from feline urine.

23. The method of claim 2, wherein the recombinant FeMV is from feline urine, feline blood, feline thymus, feline lymph nodes, feline urinary tract tissue, or feline respiratory tract tissue.

24. A method of detecting the presence of FeMV in a sample, the method comprising:
a. exposing an isolated test sample to primers that specifically hybridize to FeMV RNA and specifically hybridizing the primers to the FeMV RNA;
b. reverse transcribing the FeMV RNA to synthesize FeMV cDNA;
c. performing PCR amplification on the FeMV cDNA to produce a PCR amplicon;
d. detecting the presence of the PCR amplicon; and
e. comparing a presence of the PCR amplicon in the at least one test sample with an absence of PCR amplicon from a negative sample that lacks FeMV RNA, wherein detection of the PCR amplicon is indicative of the presence of one or more FeMV.

25. The method of claim 24, wherein the primers that specifically hybridize to the FeMV RNA comprise at least one forward primer with at least 90% identity to SEQ ID NOs: 1, 3, 7, 9, 11, 13, 15, 17, 19, 20, 22, 24, 25, 27, 30, and 33.

26. The method of claim 24, wherein the primers that specifically hybridize to the FeMV RNA comprise at least one forward primer comprising SEQ ID NOs: 1, 3, 7, 9, 11, 13, 15, 17, 19, 20, 22, 24, 25, 27, 30, and 33.

27. The method of claim 24, wherein the primers that specifically hybridize to the FeMV RNA comprise at least one reverse primer with at least 90% identity to SEQ ID NOs: 1, 2, 4, 5, 8, 12, 14, 16, 18, 6, 23, 26, 28, and 31.

28. The method of claim 24, wherein the primers that specifically hybridize to the FeMV RNA comprise at least one reverse primer comprising SEQ ID NOs: 1, 2, 4, 5, 8, 12, 14, 16, 18, 6, 23, 26, 28, and 31.
